(19) United States
(12) Patent Application Publication
(10) Pub. No.: US 2026/0314576 A1
(43) Pub. Date: Oct. 15, 2026

(54) A METHOD OF GENERATING STERILE AND MONOSEX PROGENY

(71) Applicant: CENTER FOR AQUACULTURE TECHNOLOGIES, INC., San Diego, CA (US)

(72) Inventors: Xavier Christophe LAUTH, San Diego, CA (US); John Terrell BUCHANAN, San Diego, CA (US); Takeshi UMAZUME, San Diego, CA (US)

(21) Appl. No.: 19/480,768

(22) PCT Filed: May 1, 2024
(86) PCT No.: PCT/US2024/027303

§ 371 Date: Oct. 31, 2025

Related U.S. Application Data
(60) Provisional application No. 63/463,337, filed on May 2, 2023.

(51) Int. Cl. A01K 67/0271; A01K 67/62; A01K 67/67

(52) U.S. Cl. CPC A01K 67/0271; A01K 67/62; A01K 67/67; A01K 2207/12; A01K 2217/03; A01K 2227/40; A01K 2267/02

(57) ABSTRACT

The disclosure provides an endogenous germ cell-less fish, crustacean, or mollusk having a chimeric gonad, where the chimeric gonad comprises at least one transplanted germ cell having a mutation that is present in the germline and disrupts the development and/or function of somatic gonadal cells, which can be used as broodstock. The disclosure also provides methods of making broodstock fish, crustaceans, or mollusks for use in producing sterilized or sex-determined sterilized fish, crustaceans, or mollusks, as well as the broodstock itself.

### STATEMENT OF GOVERNMENT RIGHTS

Aspects of the work described herein were supported by grant awards 2019-67030-29002 and 2018-33522-28745 from the USDA-National Institute of Food and Agriculture. The United States Government may have certain rights in these inventions.

### FIELD

The present disclosure relates generally to methods of sterilizing and sex-determining freshwater and seawater organisms.

### BACKGROUND

The following paragraphs are not an admission that anything discussed in them is prior art or part of the knowledge of persons skilled in the art.

Fish species have been genetically engineered (GE) to produce valuable pharmaceutical proteins or to incorporate advantageous traits for aquaculture. A variety of fish with improved growth rates, food conversion ratios, resistance to disease, and enhanced nutritional benefits, have been developed to address the future demand for seafood and the need to improve sustainability in the aquaculture industry. However, worldwide adoption of these GE fish is hampered by concerns over their accidental release into natural ecosystems. Cultured fish have been shown to reproduce and survive in natural environments, resulting in feral populations. Similarly, GE fish may have native relatives, raising the possibility that the genetic modifications will spread throughout the wild population and alter the native gene pool. Commercial GE fish therefore represent a potential threat to the environment and a challenge to policy makers and regulatory agencies tasked with risk-benefit evaluations.

One approach to address one or more of the aforementioned issues is to sterilize fish. The induction of triploidy is the most used and best studied approach for producing sterile fish. Generally, triploid fish are produced by applying temperature or pressure shock to fertilized eggs, forcing the incorporation of the second polar body and producing cells with three chromosome sets (3N). Triploid fish do not develop normal gonads as the extra chromosome set disrupts meiosis. At the industrial scale, the logistics of reliably applying pressure or temperature shocks to batches of eggs is complicated and carries significant costs. An alternative to triploid induced by physical treatments is triploid induced by genetics, which results from crossing a tetraploid with a diploid fish. Tetraploid fish, however, are difficulty to generate due to poor embryonic survival and slow growth. In some examples, triploid males produce some normal haploid sperm cells thus allowing males to fertilize eggs, though at a reduced efficiency. Also, in some species, negative performance characteristics have been associated with triploid phenotype, including reduced growth and sensitivity to disease.

Another approach for sterilizing fish is by hormone treatment extending over several weeks. However, in many cases, including these intensive long-term treatment processes do not have a desirable efficacy of sterility, and/or have been associated with decreased fish growth performance. Furthermore, treatments involving a synthetic steroid may result in higher mortality rates.

Another approach for sterilizing fish is by using transgenic-based technologies, which include a step of integrating a transgene that induce germ cell death or disrupts their migration patterns resulting in their ablation in developing embryos. However, transgenes are subject to position effect as well as silencing. Consequently, such approaches are subject to extended regulatory review processes before being considered acceptable for commercial use.

Improvements in generating sterile, sex-determined fish, crustaceans, or mollusks is desirable.

### INTRODUCTION

The following introduction is intended to introduce the reader to this specification but not to define any invention. One or more inventions may reside in a combination or sub-combination of the instrument elements or method steps described below or in other parts of this document. The inventors do not waive or disclaim their rights to any invention or inventions disclosed in this specification merely by not describing such other invention or inventions in the claims.

One or more of the previously proposed methods used for sterilizing freshwater and seawater organisms may result in: (1) an insufficient efficacy; (2) increased difficulty to propagate the sterility trait by, for example, having to perform genetic selection to identify a subpopulation of sterile individual, and/or repeating treatment at each generation; (3) an increase in operating costs by, for example, incorporating significant changes in husbandry practices, being untransferable across multiple species, increasing production times, increasing the percentage of sterile organisms with reduced growth and increased sensitivity to disease, increasing mortality rates of sterile organisms, or a combination thereof; (4) gene flow to wild populations and colonization of new habitats by cultured, non-native species; or (4) any combination thereof.

The present disclosure provides methods of producing sex-determined sterilized freshwater and seawater organisms by exploiting germline and somatic properties of nucleic acid molecules, for example genes, to produce sterile progeny. One or more examples of the present disclosure may: (1) increase efficacy of sterilization, by for example, allowing mass production of sterile individuals and ensuring that all individuals are completely sterile; (2) decrease operating costs by, for example, decreasing the amount of costly equipment or treatments, being commercially scalable, being transferable across multiple species, decreasing feed, decreasing production times, decreasing the percentage of organisms that attain sexually maturity, increasing the physical size of sexually mature organisms, or a combination thereof; (3) decrease gene flow to wild populations and colonization of new habitats by cultured non-native species; (4) increase culture performance by decreasing loss of energy to gonad development; or (5) any combination thereof, compared to one or more previously proposed methods used for sterilizing freshwater and seawater organisms.

The present disclosure also discusses methods of making broodstock freshwater and seawater organisms for use in producing sterilized or sex-determined sterilized freshwater and seawater organisms, as well as the broodstock itself.

The present disclosure provides an endogenous germ cell-less fish, crustacean, or mollusk having a gonad that produces gametes with a mutation causing progeny to be sterile. The present disclosure also provides methods of producing said endogenous germ cell-less fish, crustacean, or mollusk, as well as methods of breeding said endogenous germ cell-less fish, crustacean, or mollusk to produce sterile progeny or sterile sex-determined progeny. The present disclosure also provides methods of producing the gonad that produces gametes with a mutation causing progeny to be sterile, as well as the gonad itself.

The present disclosure also provides an endogenous germ cell-less fish, crustacean, or mollusk having a chimeric gonad, where the chimeric gonad comprises at least one transplanted germ cell having a mutation that is present in the germline and disrupts the development and/or function of somatic gonadal cells. The present disclosure also provides methods of producing the gonad that comprises at least one transplanted germ cell having a mutation that is present in the germline and disrupts the development and/or function of somatic gonadal cells, as well as the gonad itself.

The present disclosure also provides a method of generating an endogenous germ cell-less fish, crustacean, or mollusk, comprising the steps of: transplanting at least one germ cell having a mutation that is present in the germline and disrupts the development and/or function of somatic gonadal cells, into an endogenous germ cell-less fish, crustacean, or mollusk, producing a chimeric gonad.

The present disclosure further provides a method of generating a sterile fish, crustacean, or mollusk, comprising the steps of: breeding (i) an endogenous germ cell-less fish, crustacean, or mollusk as herein disclosed that is female with (ii) an endogenous germ cell-less fish, crustacean, or mollusk as herein disclosed that is male, to produce the sterile fish crustacean, or mollusk.

Other aspects and features of the present disclosure will become apparent to those ordinarily skilled in the art upon review of the following description of specific examples in conjunction with the accompanying figures.

### BRIEF DESCRIPTION OF THE DRAWINGS

Examples of the presently disclosed methods and organisms will now be described, by way of example only, with reference to the attached Figures.

FIG. 1 panels A to B is a flowchart showing an example of a herein disclosed method of generating endogenous germ cell-less fish, crustacean, or mollusk having a chimeric gonad and propagating a mutated line.

FIG. 2 panels A to C is a flowchart showing another example of a herein disclosed method of generating endogenous germ cell-less fish, crustacean, or mollusk having a chimeric gonad and propagating a mutated line.

FIG. 3 panels A to C is a flowchart showing another example of a herein disclosed method of generating endogenous germ cell-less fish, crustacean, or mollusk having a chimeric gonad and propagating a mutated line.

FIG. 4 is an illustration showing an example of a herein disclosed germ cell transplantation method to produce functional sperm carrying a miR202 deficient gene (miR-202−/−). No defects are found during the generation of oogonia and spermatogonia in miR202 null fish progenies obtained from heterozygous miR202 mutant parents. At maturity, miR202 mutant males and females are infertile. The miR202 is expressed in somatic cells surrounding the germ cells (Sertoli and Leydig cells) where it exerts its activity. The lack of miR202 protein causes a defective microenvironment where gamete maturation is impaired. To restore gametogenesis, a germline stem cell can be isolated from juvenile miR202 mutant and transplanted into recipient embryos depleted of their own PGCs but carrying a functional miR202 gene. Transplanted miR202−/− germ line stem cell will colonize the recipient gonad and since miR202 is dispensable for their continued development, the recipient somatic cells will nurse transplanted germ cell, restore gametogenesis and allow production of functional sperm and eggs, all of which carrying the mutant miR202 gene.

FIG. 5 panels A to D are photographs and a graph showing phenotypic characterization of miR202−/− female. FIG. 5A shows typical pictures of ovaries in the peritoneal cavity of homozygous miR202 mutant (top part) and WT control (bottom part) tilapia at 7-month of age. FIG. 5B shows urogenital papillae from miR202−/− and WT control females. FIG. 5C shows a highly magnified light microscopy image of dissected ovary. FIG. 5D shows a graphic representation of the average Gonado Somatic Indexes (GSI) from miR202 mutant female and WT controls.

FIG. 6 panels A to E are photographs and graphs showing maternal miR202 participate in PGC formation in Nile tilapia. FIGS. 6B, 6D and 6E illustrate the average number of PGCs in 4-day old embryos (≥12 embryos) from mutated females with mosaic (FIG. 6B), heterozygous (FIG. 6D) and homozygous miR202 mutation in germ cells. There is a significant difference (p≤0.01) comparing the embryos progeny from wild type control female. Vertical bars show standard deviation. FIGS. 6A and 6C top images represent 4 dpf tilapia embryo progeny of female transgenic line Tg(Zpc5:EGFP:nos 3′UTR) crossed with miR202 mutant males showing a normal PGC count. The GFP (+) germ cells (average n=40) cluster longitudinally around the anterior part of the gut. Bottom images of FIGS. 6A and 6C represent trunk regions of progenies from Tg(Zpc5:EGFP:nos3 3′UTR) female lines carrying mosaic miR202 gene mutations or heterozygous miR202 mutations showing reduced PGC count at 4 dpf. The bright dots represent GFP (+) cells (green). The white star points to mislocalized PGCs.

FIG. 7 panels A to B are illustrations of selected mutation at the miR202 loci. FIG. 7A shows the secondary structure tilapia (Oreochromis niloticus) pre miR202 as projected from forna (force-directed RNA) RNA visualization tool (Kerpedjiev, Hammer et al. 2015). FIG. 7B shows the location of the miR202 loci in Nile tilapia chromosome LG13. FIG. 7B also show the nucleotide sequence alignment of wild-type and selected mutants with deletions indicated by dashes covering the miR-202-5p region. The miR-202-5p sequence is shown in red ink and the miR-202-3p sequence is shown in green ink. The seed sequence of miR-202-5p is shown in dotted yellow box.

FIG. 8 panels A to C are photographs showing histology of representative of miR202−/− and WT control 7 months ovarian tissue. (FIGS. 8A and B) representative histology showing stage I oocytes. Bars: 100 micrometers. FIG. 8C show histological sections of control ovaries from a WT 7-month-old female.

FIG. 9 panels A to C are photographs and graphs showing phenotypic characterization of miR202^{-/-} male. FIG. 9A is typical photographs of dissected testes from homozygous miR202 mutant (miR202^{-/-}) and WT tilapia at 7-month of age. FIG. 9B is a graph showing the average Gonado Somatic Indexes of miR202^{-/-} and WT sibling male tilapia at months intervals. FIG. 9C is a graph showing the average sperm count from WT and miR202^{-/-} siblings at 7 months of age. Vertical bars represent standard deviation.

FIG. 10 panels A to D are photographs showing histological analysis of testes from miR202 mutant (FIGS. 10C and D) and WT control (FIGS. 10A and B) at 8 months of age. FIG. 10A to C show testicular structure of the whole testis, where miR202^{-/-} reveal severe depletion of testicular germ cells. FIG. 10B is a close-up view of the germinative compartment formed by Sertoli cell surrounding germ cell at different stage of development: SC, spermatocytes; ST, spermatids; and SZ, spermatozoa in the lobule lumen; The germinal compartments are surrounded by steroid-producing Leydig cells (lighter pink coloration). D) miR202^{-/-} empty tubules containing no visible germ cells but showing an enlarged wall of Leydig cells.

FIG. 11 are photographs illustrating histological sections of gonads from Elavl2^{Δ8/Δ8} recipient derived from miR202 mutant germ cells transplantation (GCT) and non-transplanted Elavl2^{Δ8/Δ8} controls. 113 days post transplantation, the recipient fish ovaries and testes were collected and fixed for further histological analysis. Germ cells are absent in non-transplanted control testes and ovaries. Normal spermatogenesis and oogenesis were observed in Germ cell transplanted (GCT) recipient indicating successful colonization, proliferation, and differentiation capacity of miR202-deficient spermatogonia and oogonia. Thus, the donor derived germ cell can undergo functional gametogenesis once provided with a permissive testicular or ovarian microenvironment.

FIG. 12 panels A to F are photographs illustrating typical morphology and histology of elavl2^{-/-} tilapia male transplanted with miR202^{-/-} germ cells (top part) or non-transplanted elavl2^{-/-} control (bottom panel). We found morphologically normal testes in the peritoneal cavity of recipient tilapia male (FIG. 12A) as opposed to the translucent testis of non-transplanted male (FIG. 12B) (Elavl2^{-/-}). FIGS. 12C and 12D close-up of box in FIGS. 12A and 12B under brightfield stereomicroscopy, showing opaque and translucent testes respectively. FIGS. 12E and 12F show Hematoxylin-eosin-stained histological sections of testes showing that donor derived miR202^{-/-} germ cell colonized, proliferated, and differentiated in the recipient elavl2^{-/-} tilapia. FIG. 12F shows non-transplanted recipient testes are germ cell free.

FIG. 13 is photographs showing typical morphology appearance of ovaries from elavl2^{-/-} tilapia female transplanted with miR202^{-/-} germ cells (top part) or non-transplanted elavl2^{-/-} control (bottom panel) at 8 months of age. We found morphologically normal ovaries in the peritoneal cavity of recipient tilapia female 8 months post transplantation. Non-transplanted elavl2^{-/-} show string like germ cell less ovaries at 7 months of age.

FIG. 14 panels A and B show illustrations and graphs of crosses between miR202^{-/-}-GCT female (chimera-gray color) with either WT male (blue color) or miR202^{-/-}-GCT male (FIGS. 14A and 14 B top parts). FIGS. 14A and 14B bottom graphs show the genotypes of their progeny by fin DNA PCR fragment sizing assay utilizing PCR primers that flank the mutation region analysis. The amplification products were sized and detected using capillary electrophoresis. Fragment PCR trace revealed a 174 bp and a 182 bp miR202 amplicons in all progenies from WT male crossed with GCT-female (FIG. 14A top part) and a single 174 bp amplicon for all progeny from miR202^{-/-}-GCT parents (FIG. 14B bottom part), revealing successful colonization of recipient gonad by donor germ cells and production of 100% miR202^{Δ8/Δ8} progeny.

FIG. 15 panels A and B are photographs showing the peritoneal cavity of male and female progeny from SSCs miR202-GCT female×miR202-GCT male at time intervals.

FIG. 16 panels A to C are illustrations showing the genomic region containing Elavl2 and Hermes genes and the selected mutations in these genes. Hermes (Rbmps) in very close proximity to Elavl2 (200 kb ~0.2 centimorgan) (FIG. 16A). We created a loss of function mutation in both gene (FIG. 16B). FIG. 16B is schematics of the tilapia Elavl2 gene and FIG. 16B is schematics of Hermes gene. Exon (E) are shown as shaded boxes. Arrows point to targeted loci. FIGS. 16B and 16C show the wild-type reference sequence of Elavl2 (SEQ ID NOs: 116 and 118) and Hermes (SEQ ID NOs: 142 and 144) with the sequence of the selected germ-line mutant alleles: 8 nucleotides deletion for Elavl2 (SEQ ID NOs: 117 and 119) and 16 nucleotides insertion for Hermes (SEQ ID NOs: 143 and 145). Both mutant alleles create truncated proteins that terminate at amino acid 40 and 61 rather than position 372 and 174 respectively. FIGS. 16B and 16C show the predicted protein sequences of WT and truncated mutant proteins in which the first 12 and 52 amino acids are identical to those of the wild-type Elavl2 and Hermes protein respectively and the following 28 and 9 amino acids are miscoded. Altered amino acids are highlighted.

FIG. 17 panels A to D are illustrations, graphs, and a photograph showing phenotypic characterization of the progeny from double heterozygous mutant Hermes^{Ins16/+}, Elavl2^{Δ8/+}. FIG. 17A shows the expected mendelian distribution of genotypes from completely linked genes (Elavl2 and Hermes) in which h represents a lethal receive Hermes mutation. FIG. 17B graphically shows the percentage of developing embryos 0-3 days post fertilization (blue columns represent the mean and vertical bar the standard deviation) and percentage of deformed embryos (~20%, yellow column). FIG. 17C is a photograph of 9 dpf hatchlings showing Hermes^{Ins16/Ins16} escapes (EEhh) having cranio-facial and body axis deformities and WT control (right image). FIG. 17D shows a QPCR melt curve plot of Elavl2 amplicons from N=64 surviving progeny allowing visualization of heterozygous (Ee) and homozygous (ee) Elavl2 mutants. We found no viable Hermes^{Ins16/Ins16} (hh) mutant in the progeny from double heterozygous parents at 1.5 month of age, confirming that Hermes KO mutant are non-viable.

FIG. 18 are photographs showing hatchlings progeny with normal pigmentation or amelanic typical of the homozygous tyr−/− albino mutation. Offspring from normally pigmented surrogate parents transplanted with donor germ cells from Tyr−/− mutant consistently exhibit albino pigmentation. Analysis of offspring pigmentation under stereomicroscopy in 3 dpf embryos obtained from mating GCT parents (Tyr+/+), which received germ cells from a Tyr−/− donor, (right panel) all display the albino phenotype. In contrast, outcrossing a surrogate female with a WT male (Tyr+/+) produce normally pigmented embryos (Left panel).

FIG. 19 panels A and B. Panel A is photographs showing dissected ovaries from 9-month-old sibling female tilapia, distinguishing between those heterozygous for the miR202 gene (fertile, with ripe ovaries) and those homozygous mutant (sterile, with ovaries arrested at the previtellogenic stage). Panel B is a box and whisker plot detailing the total body weight of two sibling groups (N=45/group): fertile (miR202+/−) and sterile (miR202−/−) females. Our analysis reveals a significant 17% increase in average body weight gain among the sterile compared to the fertile female group, with statistical significance (T-Test p<0.01).

### DETAILED DESCRIPTION

Generally, the present disclosure provides an endogenous germ cell-less fish, crustacean, or mollusk having a chimeric gonad, wherein the chimeric gonad comprises at least one transplanted germ cell having a mutation that is present in the germline and disrupts the development and/or function of somatic gonadal cells.

The present disclosure also provides a method of generating an endogenous germ cell-less fish, crustacean, or mollusk, comprising the steps of: transplanting at least one germ cell having a mutation that is present in the germline and disrupts the development and/or function of somatic gonadal cells, into an endogenous germ cell-less fish, crustacean, or mollusk, producing a chimeric gonad. The present disclosure also provides a method of generating a sterile sex-determined fish, crustacean, or mollusk. The method comprises the step of: breeding (i) a fertile female fish, crustacean, or mollusk having a homozygous mutation with (ii) a fertile male fish, crustacean, or mollusk having a homozygous mutation to produce the sterile sex-determined fish, crustacean, or mollusk. The mutation directly or indirectly disrupts spermiogenesis, and/or that directly disrupts vitellogenesis. The fertility of the fertile female fish, crustacean, or mollusk and the fertile male fish, crustacean, or mollusk have been rescued.

The present disclosure also provides a method of generating a sterile fish, crustacean, or mollusk, comprising the steps of: breeding (i) an endogenous germ cell-less fish, crustacean, or mollusk as herein disclosed that is female with (ii) an endogenous germ cell-less fish, crustacean, or mollusk as herein disclosed that is male, to produce the sterile fish crustacean, or mollusk.

The present disclosure also provides a method of generating a sterile fish, crustacean, or mollusk, comprising the steps of: breeding (i) an endogenous germ cell-less fish, crustacean, or mollusk produced by the herein disclosed methods that is female with (ii) an endogenous germ cell-less fish, crustacean, or mollusk produced by the herein disclosed method that is male, to produce the sterile fish crustacean, or mollusk.

The present disclose also provides an endogenous germ cell-less fish, crustacean, or mollusk having a chimeric gonad, wherein the chimeric gonad comprises at least one transplanted: a) Oogonial stem cell (OSC) from a homogametic female donor, such as XX, having a mutation that is present in the germline and disrupts the development and/or function of somatic gonadal cells; or b) Spermatogonial stem cell (SCC) from a homogametic male donor, such as ZZ, having a mutation that is present in the germline and disrupts the development and/or function of somatic gonadal cells.

The present disclosure also provides a method of generating an endogenous germ cell-less fish, crustacean, or mollusk, comprising the steps of: transplanting at least one: a) Oogonial stem cell (OSC) from a homogametic female donor, such as XX, having a mutation that is present in the germline and disrupts the development and/or function of somatic gonadal cells; or b) Spermatogonial stem cell (SCC) from a homogametic male donor, such as ZZ, having a mutation that is present in the germline and disrupts the development and/or function of somatic gonadal cells, into an endogenous germ cell-less fish, crustacean, or mollusk, producing a chimeric gonad.

The present disclosure also provides an endogenous germ cell-less fish, crustacean, or mollusk having a chimeric gonad, wherein the chimeric gonad comprises at least one transplanted: a) Oogonial stem cell (OSC) from a homogametic superfemale donor, such as WW, having a mutation that is present in the germline and disrupts the development and/or function of somatic gonadal cells; or b) Spermatogonial stem cell (SCC) from a homogametic supermale donor, such as YY, having a mutation that is present in the germline and disrupts the development and/or function of somatic gonadal cells.

The present disclosure also provides a method of generating an endogenous germ cell-less fish, crustacean, or mollusk, comprising the steps of: transplanting at least one: a) Oogonial stem cell (OSC) from a homogametic superfemale donor, such as WW, having a mutation that is present in the germline and disrupts the development and/or function of somatic gonadal cells; or b) Spermatogonial stem cell (SCC) from a homogametic supermale donor, such as YY, having a mutation that is present in the germline and disrupts the development and/or function of somatic gonadal cells, into an endogenous germ cell-less fish, crustacean, or mollusk, producing a chimeric gonad.

Optionally, the superfemale, such as WW, is generated by: transplanting at least one Oogonial stem cell (OSC) from a heterogametic female donor, such as WZ, having a mutation that is present in the germline and disrupts the development and/or function of somatic gonadal cells, into a male and a female endogenous germ cell-less fish, crustacean, or mollusk, producing a chimeric gonad; breeding the male endogenous germ cell-less fish, crustacean, or mollusk having a chimeric gonad with the female endogenous germ cell-less fish, crustacean, or mollusk having a chimeric gonad; and selecting a progeny that is homogametic by genotypic selection.

Optionally, the supermale, such as YY, is generated by: transplanting at least one Spermatogonial stem cell (SCC) from a heterogametic male donor, such as XY, having a mutation that is present in the germline and disrupts the development and/or function of somatic gonadal cells, into a male and a female endogenous germ cell-less fish, crustacean, or mollusk, producing a chimeric gonad; breeding the male endogenous germ cell-less fish, crustacean, or mollusk having a chimeric gonad with the female endogenous germ cell-less fish, crustacean, or mollusk having a chimeric gonad; and selecting a progeny that is homogametic by genotypic selection.

The present disclosure also provides a method of generating a sterile sex-determined fish, crustacean, or mollusk, comprising the steps of: breeding (i) an endogenous germ cell-less fish, crustacean, or mollusk as herein disclosed that is female with (ii) an endogenous germ cell-less fish, crustacean, or mollusk as herein disclosed that is male and from the same sex-determination system as the (i) female, to produce the sterile sex-determined fish, crustacean, or mollusk.

The present disclosure also provides a method of generating a sterile sex-determined fish, crustacean, or mollusk, comprising the steps of: breeding (i) an endogenous germ cell-less fish, crustacean, or mollusk produced by the herein disclosed methods that is female with (ii) an endogenous germ cell-less fish, crustacean, or mollusk produced by the herein disclosed methods that is male and from the same sex-determination system as the (i) female, to produce the sterile sex-determined fish, crustacean, or mollusk.

In the context of the present disclosure, a fish refers to any gill-bearing craniate animal that lacks limbs with digits. Examples of fish are carp, tilapia, salmon, trout, and catfish. In the context of the present disclosure, a crustacean refers to any arthropod taxon. Examples of crustaceans are crabs, lobsters, crayfish, and shrimp. In the context of the present disclosure, a mollusk refers to any invertebrate animal with a soft unsegmented body usually enclosed in a calcareous shell. Examples of mollusks are clams, scallops, oysters, octopus, squid and chitons. The fish, crustacean, or mollusk may be an Atlantic salmon, Rainbow Trout, Coho Salmon, tilapia, cobia, Seriola spp., Grouper, Snapper, barramundi, Sea Bream, Sea Bass, lumpfish, sturgeon, Litopeneus vannamei, Peneaus monadon, oysters, clams or mussels.

A sterile fish, crustacean, or mollusk refers to any fish, crustacean, or mollusk with a diminished ability to generate progeny through breeding or crossing or mixing gametes as compared to its wild-type counterpart; for example, a sterile fish, crustacean, or mollusk may have an about 50%, about 75%, about 90%, about 95%, or 100% reduced likelihood of producing viable progeny. In contrast, a fertile fish, crustacean, or mollusk refers to any fish, crustacean, or mollusk that possesses the ability to produce progeny through breeding or crossing or mixing gametes. Breeding and crossing refers to any process in which a male of a species and a female of a species mate or their gametes are mixed to produce fertile eggs, progeny or offspring.

A sex-determined fish, crustacean, or mollusk refers to any fish, crustacean, or mollusk progeny in which the sex of the progeny has been pre-determined by disrupting the progeny's sexual differentiation pathway. In some examples, sex-determined progeny of the same generation are monosex.

Chimeric gonad refers to a gonad that has been colonized with one or more germ cells from a genotype distinct from the genotype housing the gonad. Germ cells refers to any biological cell that gives rise to the gametes of an organism. The germline refers to a population germ cells that pass on their genetic material to the progeny. Somatic cells refers to any cell that is not a gamete, germ cell, or stem cell, and therefore does not pass through generations. Somatic gonadal cells refer to somatic cells that generate the various cell types within the testis or ovary that support gametogenesis.

A mutation present in the germline and disrupts the development and/or function of somatic gonadal cells refers to any genetic mutation of a nucleic acid molecule that is passed from one generation to another and that directly or indirectly somatically modulates gonadal development and/or function. Directly or indirectly modulating refers to: (1) mutating the coding sequence of one or more nucleic acid molecules that is passed from one generation to another and that directly or indirectly somatically modulates gonadal development and/or function; (2) mutating a non-coding sequence that has at least some control over the transcription of one or more nucleic acid molecules that is passed from one generation to another and that directly or indirectly somatically modulates gonadal development and/or function; (3) mutating the coding sequence of another gene or nucleic acid molecule that is involved in post-transcriptional regulation of one or more nucleic acid molecules that is passed from one generation to another and that directly or indirectly somatically modulates gonadal development and/or function; or (4) any combination thereof. A mutation may be any type of alteration of a nucleotide sequence of interest, for example, nucleotide insertions, nucleotide deletions, and nucleotide substitutions. The mutation that is present in the germline and disrupts the development and/or function of somatic gonadal cells may be a mutation in microRNA miR202 or an ortholog thereof.

Somatically modulating gonadal development and/or function refers to disrupting the maturation of gonadal somatic cells and/or disrupting the interaction between gonadal somatic cells and germ cells in the gonad. Examples include disrupting gametogenesis; disrupting the development and/or function of testis and/or ovary cells; disrupting the development and/or function of Sertoli, Theca, Granulosa, and/or Leydig cells; disrupting at least one signaling molecule produced by Sertoli, Theca, Granulosa, and/or Leydig cells; disrupting at least one secreted diffusible signal protein or growth factor protein that regulates Spermatogonial stem cell (SCC) and/or Oogonial stem cell (OSC) renewal and/or differentiation. The at least one secreted diffusible signal protein or growth factor protein may be glial cell line-derived neurotrophic factor (GDNF); bone morphogenetic protein 4 (BMP4); stem cell factor (SCF); fibroblast growth factor 2 (FGF2); C-X-C motif chemokine 12 (CXCL12); and/or epidermal growth factor (EGF).

Germ cell transplantation refers to any process in which one or more stem cells from a fish, crustacean, or mollusk is transplanted into another fish, crustacean, or mollusk. In some examples according to the present disclosure, the germ stem cell transplantation is a process comprising: obtaining one or more germline stem cells, for example from about 1 to about 6,000 germ cells, or from about 500 to about 6,000 germ cells, from a fertile homozygous male or female fish, crustacean, or mollusk having a mutation that is present in the germline and disrupts the development and/or function of somatic gonadal cells; and transplanting the one or more germline stem cells into an endogenous germ cell-less recipient male or female fish, crustacean, or mollusk. A recipient male or female fish, crustacean, or mollusk is any embryo depleted of their own germ cells but homozygous wild type in the germline at the locus for the mutation that is present in the donor male or female fish, crustacean, or mollusk. Optionally, the donor male or female fish, crustacean, or mollusk can be a juvenile or adult fish. A juvenile fish, crustacean, or mollusk is a stage of maturation that is prior to development and/or production of hormones related to sexual maturation or puberty; is at an age of from about 1 month to about 9 months, for example, from about 6 months to about 9 months; and/or has a gonadosomatic index (GSI) of from about 0.1% to about 0.5%. The recipient after transplantation is a fish, crustacean or mollusk having a chimeric gonad with normal somatic cells but a mutant germline. Optionally, the at least one germ cell is transplanted into the peritoneal cavity of the embryo or hatchling of the recipient, for example, the endogenous germ cell-less fish, crustacean, or mollusk.

A germ cell-less recipient may be created using genetic mutation, ploidy manipulation, such as triploidy manipulation, hybridization strategies, exposure to high levels of sex hormones, using morpholinos to disrupt primordial germ cell development, using sterile hybrids, exposure to chemicals and high temperature. See examples described in: Hunter et al, 1982; Solar et al, 1984; Piferrer et al, 1994; Hunter, G. A., E. M. Donaldson, F. W. Goetz, and P. R. Edgell. 1982. Production of all-female and sterile Coho salmon, and experimental evidence for male heterogamety. Transactions of the American Fisheries Society 111: 367-372; Piferrer, F, M Carillo, S. Zanuy, I. I. Solar, and E. M. Donaldson. 1994. Induction of sterility in Coho salmon (Oncorhynchus kisutch) by androgen immersion before first feeding. Aquaculture 119: 409-423; Solar, I., E. M. Donaldson, and G. A. Hunter. 1984. Optimization of treatment regimes for controlled sex differentiation and sterilization in wild rainbow trout (Salmo gairdeneri Richardson) by oral administration of 17α-methyltestosterone. Aquaculture 42: 129-139; and PCT Publication No. WO2020/033940. For Interspecific hybridization, see examples described in: Saito, T.; Goto-Kazeto, R.; Arai, K.; Yamaha, E. Xenogenesis in Teleost Fish through Generation of Germ-Line Chimeras by Single Primordial Germ Cell Transplantation. Biol. Reprod. 2008, 78, 159-166; Yoshikawa, Hiroyuki, et al. “Hybrid sterility in fish caused by mitotic arrest of primordial germ cells.” Genetics 209.2 (2018): 507-521. Yoshikawa, H.; Xu, D.; Ino, Y.; Yoshino, T.; Hayashida, T.; Wang, J.; Yazawa, R.; Yoshizaki, G.; Takeuchi, Y. Hybrid Sterility in Fish Caused by Mitotic Arrest of Primordial Germ Cells. Genetics 2018, 209, 507-521; Xu, D.; Yoshino, T.; Konishi, J.; Yoshikawa, H.; Ino, Y.; Yazawa, R.; Dos Santos Nassif Lacerda, S. M.; De França, L. R.; Takeuchi, Y. Germ Cell-Less Hybrid Fish: Ideal Recipient for Spermatogonial Transplantation for the Rapid Production of Donor-Derived Sperm. Biol. Reprod. 2019, 101, 492-500. For Triploidization, see examples described in: Lee, S.; Bang, W. Y.; Yang, H. S.; Lee, D. S.; Song, H. Y. Production of Juvenile Masu Salmon (Oncorhynchus masou) from Spermatogonia-Derived Sperm and Oogonia-Derived Eggs via Intraperitoneal Transplantation of Immature Germ Cells. Biochem. Biophys. Res. Commun. 2021, 535, 6-11, Seki, S.; Kusano, K.; Lee, S.; Iwasaki, Y.; Yagisawa, M.; Ishida, M.; Hiratsuka, T.; Sasado, T.; Naruse, K.; Yoshizaki, G. Production of the Medaka Derived from Vitrified Whole Testes by Germ Cell Transplantation. Sci. Rep. 2017, 7, 43185; Yoshikawa, H.; Takeuchi, Y.; Ino, Y.; Wang, J.; Iwata, G.; Kabeya, N.; Yazawa, R.; Yoshizaki, G. Efficient Production of Donor Derived Gametes from Triploid Recipients Following Intra-Peritoneal Germ Cell Transplantation into a Marine Teleost, Nibe Croaker (Nibea mitsukurii). Aquaculture 2017, 478, 35-47. For Dnd-Morpholino, see examples described in: Saito, T.; Goto-Kazeto, R.; Arai, K.; Yamaha, E. Xenogenesis in Teleost Fish through Generation of Germ-Line Chimeras by Single Primordial Germ Cell Transplantation. Biol. Reprod. 2008, 78, 159-166. Franek, R.; Kaspar, V.; Shah, M. A.; Gela, D.; Pšeni ̌cka, M. Production of Common Carp Donor-Derived Offspring from Goldfish Surrogate Broodstock. Aquaculture 2021, 534, 736252. Yoshizaki, G.; Takashiba, K.; Shimamori, S.; Fujinuma, K.; Shikina, S.; Okutsu, T.; Kume, S.; Hayashi, M. Production of Germ Cell-Deficient Salmonids by dead end Gene Knockdown, and Their Use as Recipients for Germ Cell Transplantation. Mol. Reprod. Dev. 2016, 83, 298-311. For Dnd-knockout, see examples described in: Yoshizaki, G. Germ Cell Transplantation in Fish: Mutant dnd Rainbow Trout Can Produce Chinook Salmon Gametes. In Proceedings of the 2021 Exotic Species Webinar Series Recordings, 10 Feb. 2021; Society for the Study of Reproduction: Reston, VA, USA, 2021. Li, Q.; Fujii, W.; Naito, K.; Yoshizaki, G. Application of dead end-Knockout Zebrafish as Recipients of Germ Cell Transplantation. Mol. Reprod. Dev. 2017, 84, 1100-1111. For Chemical and high temperature, see examples described in: Chemical with high temperature: 100,32,72 Majhi, S. K.; Hattori, R. S.; Rahman, S. M.; StrOssmann, C. A. Surrogate Production of Eggs and Sperm by Intrapapillary Transplantation of Germ Cells in Cytoablated Adult Fish. PLoS ONE 2014. Ren, Y.; Sun, Z.; Wang, Y.; Yu, Q.; Wang, G.; He, Z.; Liu, Y.; Jiang, X.; Kang, X.; Hou, J. Production of Donor-Derived Offsprings by Allogeneic Transplantation of Oogonia in the Adult Japanese Flounder (Paralichthys olivaceus). Aquaculture 2021, 543, 736977. Lacerda, S. M. S. N.; Batlouni, S. R.; Costa, G. M. J.; Segatelli, T. M.; Quirino, B. R.; Queiroz, B. M.; Kalapothakis, E.; França, L. R. A New and Fast Technique to Generate Offspring after Germ Cells Transplantation in Adult Fish: The Nile Tilapia (Oreochromis niloticus) Model. PLoS ONE 2010. Each of the references in this paragraph is incorporated by reference. In examples of creating a germ cell-less recipient using genetic mutation, the recipient may have a null mutation in dnd1, Elavl2, vasa, nanos3, and/or piwi-like gene.

The at least one germ cell may be a Spermatogonial stem cell (SCC); an Oogonial stem cell (OSC). The at least one germ cell may be from a heterogametic male donor, such as XY; from a homogametic female donor, such as XX; from a homogametic male donor, such as ZZ; from a heterogametic female donor, such as WZ; from a homogametic superfemale donor, such as WW; or from a homogametic supermale donor, such as YY.

In some examples when producing sterile fish, crustaceans, or mollusks progeny is preferable, at least one herein disclosed Oogonial stem cell (OSC) from a female donor, is transplanted into a herein disclosed endogenous germ cell-less fish, crustacean, or mollusk that is female, and into a herein disclosed endogenous germ cell-less fish, crustaceans, or mollusk that is male; and the male and female recipient fish, crustacean or mollusks are bred producing sterile fish, crustaceans, or mollusks.

In some other examples when producing sterile fish, crustaceans, or mollusks progeny is preferable, at least one herein disclosed Spermatogonial stem cell (SCC) from a male donor, is transplanted into a herein disclosed endogenous germ cell-less fish, crustacean, or mollusk that is female, and into a herein disclosed endogenous germ cell-less fish, crustaceans, or mollusk that is male; and the male and female recipient fish, crustacean or mollusks are bred producing sterile fish, crustaceans, or mollusks.

In some examples when producing sterile female fish, crustaceans, or mollusks progeny is preferable, at least one herein disclosed Oogonial stem cell (OSC) from a homogametic female donor, such as XX, is transplanted into a herein disclosed endogenous germ cell-less fish, crustacean, or mollusk that is female, and into a herein disclosed endogenous germ cell-less fish, crustaceans, or mollusk that is male; and the male and female recipient fish, crustacean or mollusks are bred producing sterile fish, crustaceans, or mollusks that are female.

In other examples, when producing sterile female fish, crustaceans, or mollusks progeny is preferable, at least one herein disclosed Oogonial stem cell (OSC) from a homogametic superfemale donor, such as WW, is transplanted into a herein disclosed endogenous germ cell-less fish, crustacean, or mollusk that is female, and into a herein disclosed endogenous germ cell-less fish, crustacean, or mollusk that is male; and the male and female recipient fish, crustaceans or mollusks are bred producing sterile fish, crustaceans, or mollusks that are female. The superfemale, such as WW, may be generated by: transplanting at least one herein disclosed Oogonial stem cell (OSC) from a heterogametic female donor, such as WZ into a herein disclosed male and female endogenous germ cell-less fish, crustaceans, or mollusks, producing a chimeric gonad; breeding the male endogenous germ cell-less fish, crustacean, or mollusk having a chimeric gonad with the female endogenous germ cell-less fish, crustacean, or mollusk having a chimeric gonad; and selecting a progeny that is homogametic by genotypic selection.

In some examples when producing sterile male fish, crustaceans, or mollusks progeny is preferable, at least one herein disclosed Spermatogonial stem cell (SCC) from a homogametic male donor, such as ZZ, is transplanted into a herein disclosed endogenous germ cell-less fish, crustacean, or mollusk that is female, and into a herein disclosed endogenous germ cell-less fish, crustacean, or mollusk that is male; and the male and female recipient fish, crustacean or mollusks are bred producing sterile fish, crustaceans, or mollusks that are male.

In other examples when producing sterile male fish, crustaceans, or mollusks progeny is preferable, at least one herein disclosed Spermatogonial stem cell (SCC) from a homogametic supermale donor, such as YY, is transplanted into a herein disclosed endogenous germ cell-less fish, crustacean, or mollusk that is female, and into a herein disclosed endogenous germ cell-less fish, crustacean, or mollusk that is male; and the male and female recipient fish, crustaceans or mollusks are bred producing sterile fish, crustaceans, or mollusks that are male. The supermale, such as YY, may be generated by: transplanting at least one herein disclosed Spermatogonial stem cell (SCC) from a heterogametic female donor, such as XY into a herein disclosed male and female endogenous germ cell-less fish, crustaceans, or mollusks, producing a chimeric gonad; breeding the male endogenous germ cell-less fish, crustacean, or mollusk having a chimeric gonad with the female endogenous germ cell-less fish, crustacean, or mollusk having a chimeric gonad; and selecting a progeny that is homogametic by genotypic selection.

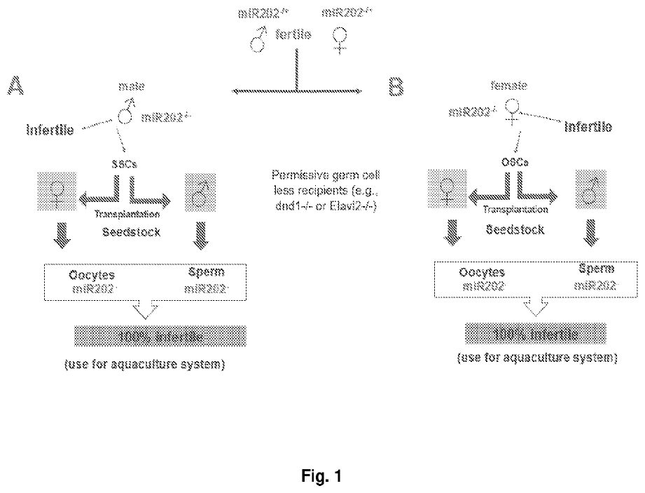

FIG. 1 is a flowchart showing an example of a herein disclosed method of generating endogenous germ cell-less fish, crustacean, or mollusk having a chimeric gonad and propagating a mutated line, i.e., generating a sterile fish, crustacean, or mollusk therefrom. The mutant donor has a mutation that is present in the germline and disrupts the development and/or function of somatic gonadal cells. In this example, the mutation is in miR202. The mutation causes a defect in the microenvironment or niche of the testis and ovary resulting in germ cell development arrest and infertility in adult homozygous mutant males and females (FIG. 1A). To develop males and females that only produce oocytes and sperm carrying the mutation, a chimera using germ cell transplantation is produced. To produce the chimera, ovarian or testicular cell suspensions obtained from a homozygous mutant fish, crustacean, or mollusk is transplanted into the peritoneal cavity of an endogenous germ cell-less recipient embryo or hatchling of a fish, crustacean, or mollusk that are wild type for the mutation producing a chimeric gonad. The host chimeric recipient has normal somatic cells and a mutant germ line. In the chimeric recipients, the mutation is silent and does not block germ cell development. The chimeric recipients have ovaries and testes capable of nurturing mutant germ cells and only produce donor derived gametes carrying the mutated genes (FIG. 1B). The recipient fish, crustacean, or mollusk can be used as commercial broodstock for mass production of sterile fish, crustaceans or mollusks.

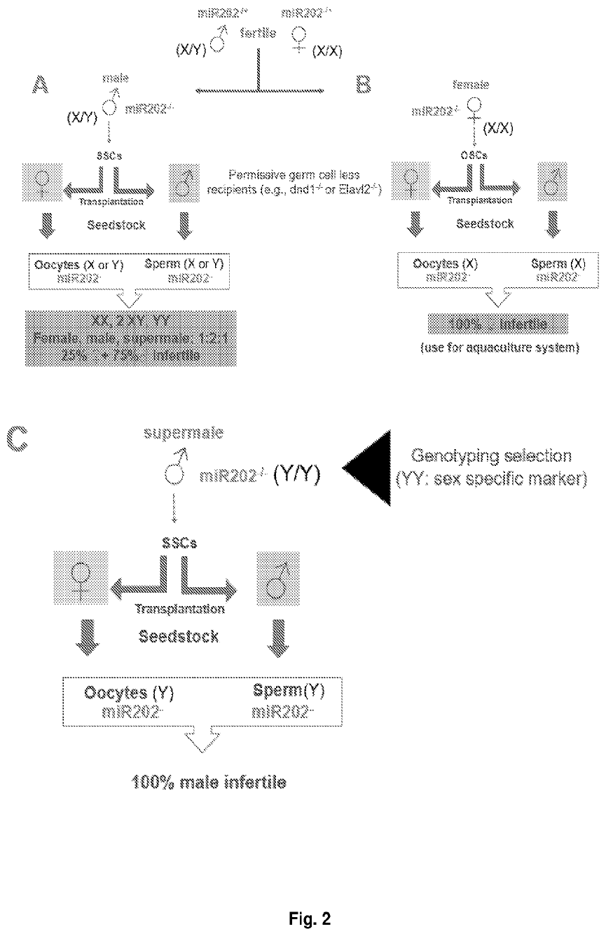

FIG. 2 is a flowchart showing an example of a herein disclosed method of generating endogenous germ cell-less fish, crustacean, or mollusk having a chimeric gonad and propagating a mutated line, i.e., generating monosex sterile fish, crustacean, or mollusk. The mutant donor has a mutation that is present in the germline and disrupts the development and/or function of somatic gonadal cells. In this example, the mutation is in miR202. When Spermatogonial Stem Cells (XY) from the donor are transplanted into recipients, only X and Y gametes will be produced resulting in sperm and eggs containing X and Y chromosomes (FIG. 2A). When Oogonial Stem Cells (OSCs) are transplanted from the donor, only X gametes will be produced resulting in sperm and eggs containing X chromosomes. Incrossing recipients will produce offspring having different male-to-female sex ratio: following SSC transplantation, Y eggs will be produced, leading to XX, XY and YY at a mendelian distribution of 1:2:1 ratio; and following OSCs (XX) transplantation, male and female producing gametes containing X chromosome only will be produced. Incrossing germ cell transplanted recipients derived from OSCs will result exclusively in female offspring production (FIG. 2B). When SSCs (YY) are transplanted, only Y gametes will be produced resulting in sperm and eggs containing Y chromosomes. Incrossing recipients from this group will produce 100% male infertile progeny (FIG. 2C). In some examples, the herein disclosed propagating and incrossing may be performed by mixing sperm and eggs.

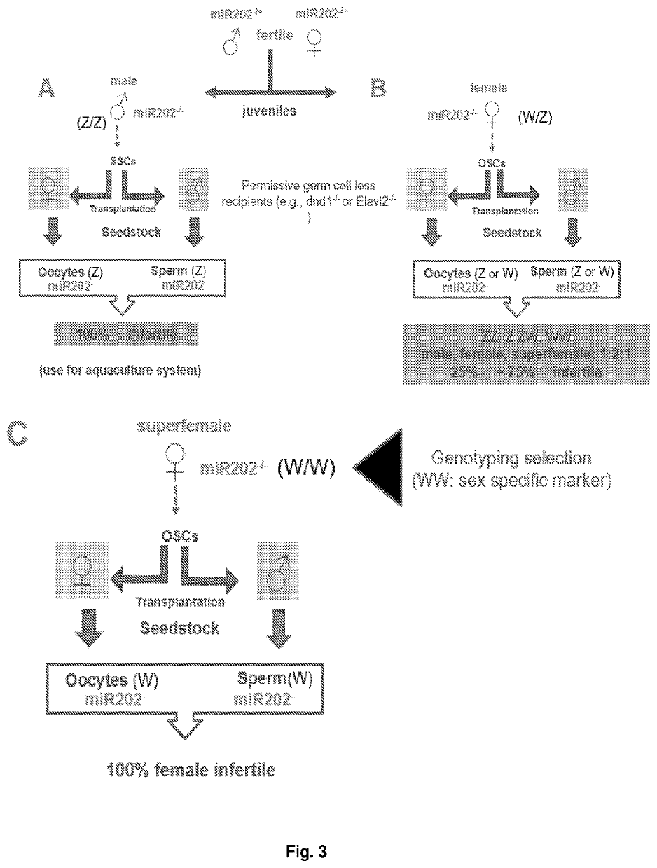

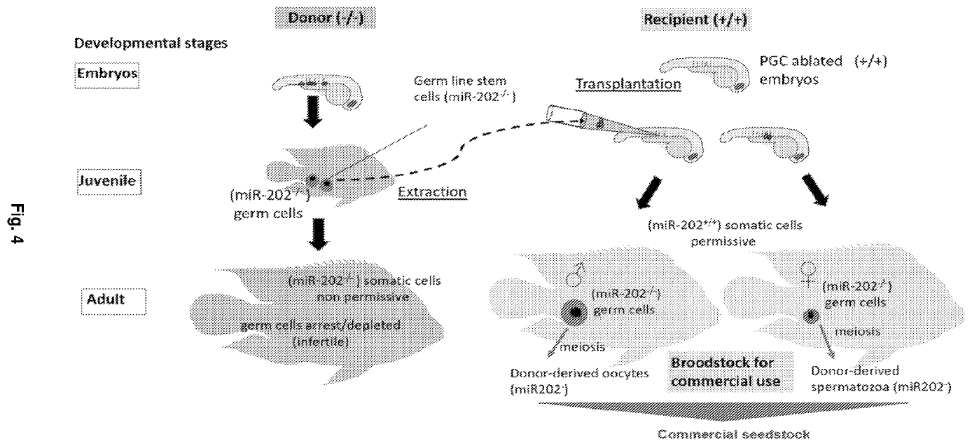

FIG. 3 is a flowchart showing an example of a herein disclosed method of generating endogenous germ cell-less fish, crustacean, or mollusk having a chimeric gonad and propagating a mutated line, i.e., generating monosex sterile fish, crustacean, or mollusk. The mutant donor has a mutation that is present in the germline and disrupts the development and/or function of somatic gonadal cells. In this example, the mutation is in miR202. When Spermatogonial Stem Cells (ZZ) from the donor are transplanted into recipients, only Z gametes will be produced resulting in sperm and eggs containing Z chromosomes (FIG. 3A). When Oogonial Stem Cells (OSCs) are transplanted, Z and W gametes will be produced resulting in sperm and eggs containing either Z or W chromosomes. Incrossing germ cell transplanted recipients will produce offspring having different male-to-female sex ratio: following OSC transplantation, W sperm will be produced, leading to ZZ, ZW and WW at a mendelian distribution of 1:2:1 ratio, resulting in 75% female progeny and following SSCs (ZZ) transplantation, male and female producing gametes containing Z chromosome only will be produced. Incrossing ZZ germ cell transplanted recipients derived from SSCs will result exclusively in male offspring production (FIG. 3A). When OSCs from superfemale (WW) are transplanted, only W gametes will be produced, resulting in sperm and eggs containing W chromosomes. Incrossing recipients from this group will produce female only progeny (FIG. 3C). In some examples, the herein disclosed propagating and incrossing may be performed by mixing sperm and eggs.

The present inventors surprisingly found that miR202 mutant tilapia lines developed penetrant male and female infertility phenotypes. Considering that the same gene mutation did not impair zebrafish fertility and only caused partial infertility in medaka, the high level of infertility described here was unexpected.

The present inventors further discovered that despite the existence of mRNA targets for miR202 regulation within germ cells, infertility in tilapia resulted from the loss of miR202 function in somatic gonadal cells. These miR202 properties offered the possibility to mass produce infertile tilapia. The inventor's results show for the first time how a single gene mutation combined with germ cell transplantation allows mass production of infertile fish, crustaceans, or mollusks of predictable specific gender.

### EXAMPLES

### Example 1—Materials and Methods

Animal used and ethical statement: All experiments complied with US regulations ensuring animal welfare and animal husbandry procedures were performed according to IACUC-approved animal protocol CAT-004. Tilapia (Oreochromis niloticus) lines used in this study are derived from a Brazilian strain and carry a transgene where an oocyte specific promoter was functionally linked to a “eGFP:nos3 3′UTR” cassette. The resulting transgenic female line express the Green Fluorescent Protein (GFP) in the PGCs of their offspring embryos. Tilapia were housed in multiple recirculating aquatic holding systems to accommodate all life stages. The culture system was maintained at 27° C. (12H light: 12H dark).

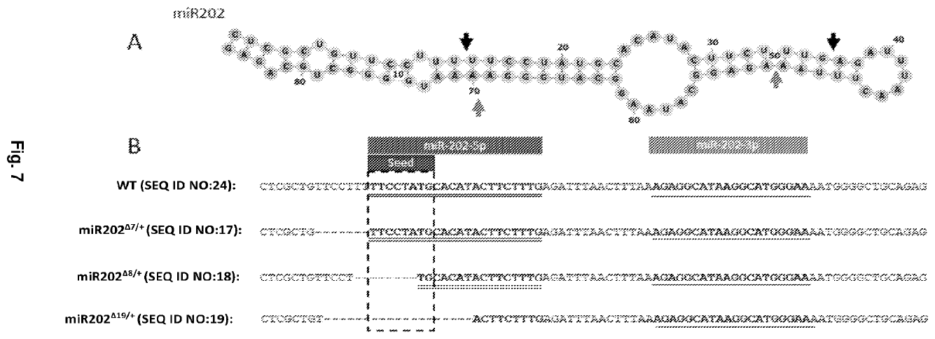

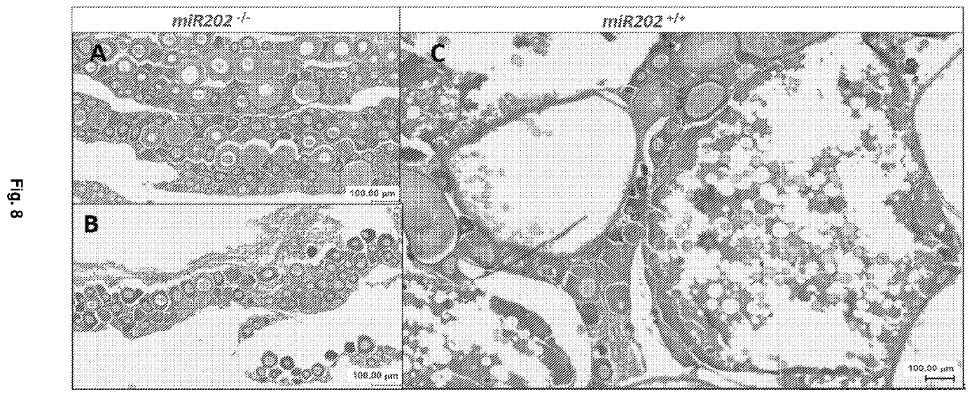

Gene Editing: The miR202 KO tilapia were generated with genome editing technology. The tilapia miR202 gene (MiRBase; Ensemble: ENSONIG00000021943) is located in an intergenic region on the Linkage group 13 (putative chromosome). The flanking sequence 5′-GTATGTGCATAGGAAAA-3′ was selected as a single guide RNA to target the tilapia miR-202-5p seed sequence. These founders were genotyped by PCR fragment analysis. Tilapia lines carrying a 7, 8 and 19-bp deletions (TTCCTTT and TTTTCCTA and TCCTTTTTCCTATGCACAT) around the miR202 seed sequence were selected and bred for this project (see FIG. 7).

Fluorescence PCR for F0 genotyping: PCR reactions used 3.8 μL of water, 0.2 μL of fin-DNA and 5 μL of PCR master mix (Quiagen Multiplex PCR) with 1 ul of primer mix consisting of the following three primers: the Labeled tail primer with fluorescent tag (6-FAM, NED), amplicon-specific forward primer with forward tail (5′-TGTAAAACGACGGCCAGT-3′ and 5′-TAGGAGTGCAGCAAGCAT-3′) amplicon-specific reverse primer (5′-GTTCCAGTGTCCAGAATCGGG-3′ and 5′-CTGGTGGAATACCTCTGC-3′). PCR conditions were as follows: denaturation at 95° C. for 15 min, followed by 30 cycles of amplification (94° C. for 30 sec, 57° C. for 45 sec, and 72° C. for 45 sec), followed by 8 cycles of amplification (94° C. for 30 sec, 53° C. for 45 sec, and 72° C. for 45 sec) and final extension at 72° C. for 10 min, and an indefinite hold at 4° C. One-two microliters of 1:10 dilution of the resulting amplicon were resolved via capillary electrophoresis (CE) with an added LIZ labeled size standard to determine the amplicon sizes accurate to base-pair resolution (Retrogen Inc., San Diego). The raw trace files were analyzed on Peak Scanner software (ThermoFisher). The size of the peak relative to the wild-type peak control determines the nature (insertion or deletion) and length of the mutation. The number of peak(s) indicate the level of mosaicism. We selected F0 mosaic founder carrying the fewest number of mutant alleles (2-4 peak preferentially). The allele sizes were used to calculate the observed indel mutations. Mutations that are not in multiples of 3 bp and thus predicted to be frameshift mutations were selected for further confirmation by sequencing except for mutation in the non-coding sequence of genes targeted. Mutations of size greater than 8 bp but smaller than 30 bp were preferentially selected to ease genotyping by QPCR melt analysis for subsequent generations. For sequence confirmation, the PCR product of the selected indel is further submitted to sequencing. Sequencing chromatography of PCR showing two simultaneous reads are indicative of the presence of indels. The start of the deletion or insertion typically begins when the sequence read becomes divergent. The dual sequences are then carefully analyze to detect unique nucleotide reads. The pattern of unique nucleotide read is then analyzed against series of artificial single read patterns generated from shifting the wild type sequence over itself incrementally.

Quantitation of PGC Number in Early Embryos: In the transgenic line, Tg(Zpc5:eGFP:tnos 3′UTR) the tilapia Zpc5 promoter is an oocyte-specific promoter, active during oogenesis prior to the first meiotic division. As such, all embryos from a heterozygous or homozygous transgenic female inherit the eGFP:tnos 3′UTR mRNA, which localizes and becomes expressed exclusively in PGCs through the action of cis-acting RNA elements in their 3′UTR (tilapia nos3 3′UTR). Embryos (4 days post fertilization) were euthanized by an overdose of tricaine methanesulfonate (MS-222, 200-300 mg/l) by prolonged immersion for at least 10 minutes. Stock preparation is 4 g/L 10 buffered to pH 7 in sodium bicarbonate (at 2:1 bicarb to MS-222). The embryos were transferred onto a glass surface in PBS and their yolk removed. Deyolked embryos were squashed between a microscope slide and a cover slip and analyzed under fluorescent microscopy equipped with camera for imaging. Images were taken using either bright field or epifluorescent light with an enhanced green fluorescent protein filter.

F1 genotyping: The selected male founders were crossed with tilapia female carrying the ZPC5:eGFP:tnos 3′UTR construct. Their F1 progeny were raised to 2 months of age, anesthetized by immersion in 200 mg/L MS-222 (tricaine) and transferred onto a clean surface using a plastic spoon. Their fin was clipped with a razor blade, and placed onto a well (96 well plate with caps). Fin clipped fish were then placed in individual jars while their fin DNA was analyzed by fluorescence PCR. In brief, 60 μl of a solution containing 9.4% Chelex and 0.625 mg/ml proteinase K is added to each well for overnight tissue digestion and gDNA extraction in a 55° C. incubator. The plate is then vortexed and centrifuged. gDNA extraction solution was then diluted 10× with ultra-clean water to remove any PCR inhibitors in the mixture. Typically, we analyzed 80 juveniles/founder to select and raised batches of approximately 20 juveniles carrying identical size mutations. In some examples, the herein disclosed crossing may be performed by mixing sperm and eggs.

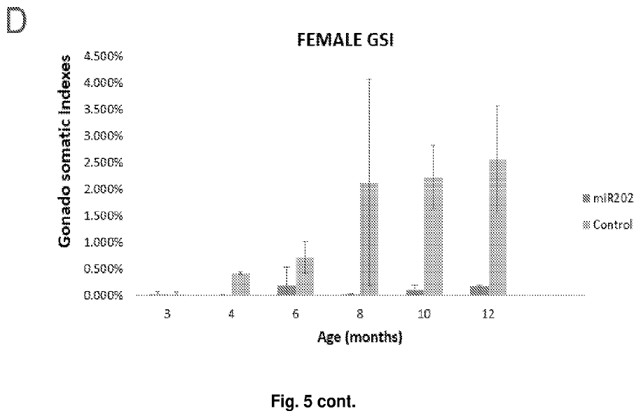

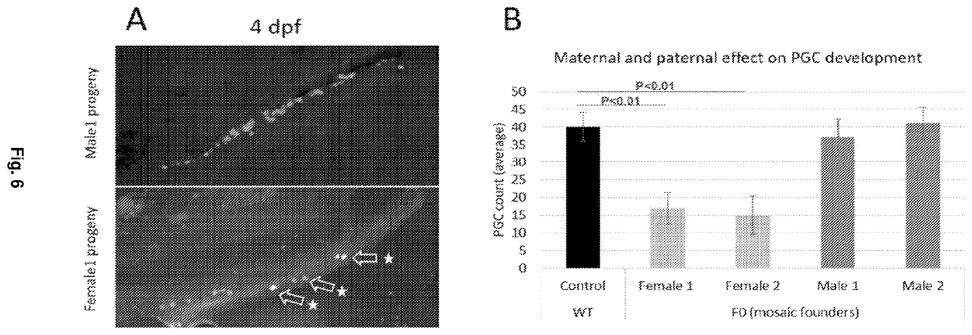

QPCR genotyping of F1 and F2 generations: Real-time qPCR was performed on a ROTOR-GENE RG-3000 REAL TIME PCR SYSTEM (Corbett Research). 1-μL genomic DNA (gDNA) template (diluted at 5-20 ng/μl) was used in a total volume of 10 μL containing 0.15 μM concentrations each of the forward and reverse primers and 5 μL of QPCR 2× Master Mix (Apex Bio-research products). qPCR primers used were 5′-GTTCCAGTGTCCAGAATCGGG-3′ and 5′-CTGGTGGAATACCTCTGC-3′. The qPCR was performed using 40 cycles of 15 seconds at 95° C., 60 seconds at 60° C., followed by melting curve analysis to confirm the specificity of the assay (67° C. to 97° C.). In this approach, short PCR amplicons (approximately 120-200 bp) that include the region of interest are generated from a gDNA sample, subjected to temperature-dependent dissociation (melting curve). When induced indels are present in heterozygote gDNA, heteroduplex as well as different homoduplex molecules are formed. The presence of multiple forms of duplex molecules is detected by Melt profile, showing whether duplex melting acts as a single species or more than one species. Generally, the symmetry of the melting curve and melting temperature infers on the homogeneity of the dsDNA sequence and its length. Thus, homozygous and wild type (WT) show symmetric melt curves that are distinguishable by varied melting temperature. The Melt analysis was performed by comparison with reference DNA sample (from control wild type DNA) amplified in parallel with the same master mix reaction. In short, variation in melt profile distinguishes amplicons generated from homozygous, heterozygote and WT gDNA (FIG. 5).

Assessment of sterility in males: The volume of strippable sperm and sperm density was measured from 10 males (5 months of age) for each genotype. Sperm were counted using a Neubauer hemocytometer slide, as well as by spectrophotometry (optical density (O.D) at 600 nm) of serially diluted samples. Sperm motility was measured in terms of percent motile spermatozoa in field of view [1]. Morphology of the sperm cells stained with eosin-nigrosin was analyzed under light microscopy at 400×. Fertilization capacity of sperm was assayed by in vitro fertilization of wild type eggs from 3 different females at the optimal sperm to egg ratio (100 eggs for 5.106 spermatozoa). Wild type egg quality was tested in parallel using sperm from WT males. Fertilization rates were expressed as a percentage of surviving embryos to total eggs collected at 24 hrs post fertilization. The mean values obtained from these studies were compared across mutant genotypes using an unpaired t-test.

Assessment of sterility in females: We recorded the body weight of all fish sampled. A minimum of six females for each genotype was dissected at 4 and 6 months of age and their gonads photographed in situ before dissection. The mean total gonadosomatic index was statistically compared across all genotypes (unpaired T-test). Survival of eggs, embryos and larvae produced from a minimum of six mutant females outcrossed with wild-type males were statistically analyzed (unpaired T-test) and compared to controls (wild-type females crossed with mutant males). In some examples, the herein disclosed outcrossing may be performed by mixing sperm and eggs.

Histology: The fixed samples were dehydrated and embedded in paraffin; 5-μm-thick (paraffin) sections were cut, and the paraffin sections were stained with hematoxylin-eosin for histological examination with a light microscope [2].

Donor cell isolation and germ cell transplantation: Germ cell stem cells were harvested from the gonads of 3-4 months old fish (~50-70 g) through enzymatic digestion as described by Lacerda [3]. In brief, the freshly isolated gonads were minced and incubated in 1 ml of 0.5% trypsin (Worthington Biochemical Corp., Lakewood, NJ) in PBS (pH 8.2) containing 5% fetal bovine serum (Gibco Invitrogen Co., Grand Island, NY) and 0.05% DNase I (Roche Diagnostics, Mannheim, Germany) for 3-4 h at 25° C. During incubation, gentle pipetting was applied to physically disrupt any remaining intact portions of the gonads. The resulting cell suspension was filtered through a nylon screen with a pore size of 42 μm (N-No. 330T; Tokyo Screen Co. Ltd., Tokyo, Japan) to remove any undissociated cell clumps and then resuspended in L-15 medium (Gibco Invitrogen Co.) before storage on ice until transplantation.

Hapas spawning experiments: In each hapa, three wildtype (WT) females 9-12 months of age with an average body weight of 700 g were paired with one male chosen from two genotypes (miR202+/− and miR202−/−), all originating from heterozygous parents and possessing an average body weight of 800 g at 12 mpf (mpf: months post fertilization). The fish were permitted to engage in natural spawning for a maximum period of 30 days. Monitoring of female fish occurred every other day to detect the presence of eggs in the buccal cavity. Upon discovery, females carrying eggs were captured, and the eggs were extracted from their mouths. The pit tag number of each egg-bearing female was recorded, and they were subsequently replaced with new WT females. The total number of eggs from each spawn was tallied, and the fertile embryos were then incubated in petri dishes until reaching stages 14-16 during the pharyngula period. For each spawn, the ratio of the total number of eggs to live embryos at the pharyngula stage was recorded.

Generation of germ cell free recipient: Germ cell-free recipient larvae (5-7 dpf) were anesthetized with 0.01% ethyl 3-aminobenzoate methanesulfonate salt (Sigma-Aldrich Inc.) and transferred to a Petri dish coated with 2% agar. Transplantation needles were prepared by pulling glass capillaries using an electric puller (PB-7, Narishige). The tips of the needles were sharpened with a grinder (EG-4, Narishige) until the opening reached 30 μm. Cell transplantation was performed by injecting approximately 15,000 testicular cells into the peritoneal cavity of approximately 100 larvae progeny from Elavl2 Hermes heterozygote or Dnd1 heterozygote mutant parents. After transplantation, recipient larvae were transferred back to aerated embryo hatching water and raised to adulthood.

Grow out studies: To generate groups used for growth performance trials, embryos from single cross miR202−/− oogonia GCT-male×miR202−/− oogonia GCT-female and miR20−/−2 oogonia GCT-female×WT male (XY, miR202+/+) were produced. Treatment (miR202−/−) and control (miR202+/−) embryos were reared separately using established hatchery procedures. At 2 months of aged, control fish (miR202+/−) were sexed based on the structure of their urogenital orifice and male were removed. Only female fish were found in the miR202−/− test group, as expected from their XX sex chromosome set. All fish were PIT tagged and placed together in a 1000 L tanks (total of 100 fish, with 50 fish/group). All fish will be fed three times daily, to satiation. Each fish will be individually weighed monthly over a period necessary to reach market size (~600 g-700 g, 9 months). At the end of the experiment, fish were sacrificed their individual total body weights was recorded. We further recorded the individual weights of dissected ovaries for calculation of gonadosomatic index (GSI). In some examples, the herein disclosed cross may be performed by mixing sperm and eggs.

### Example 2—Generation of Founder Lines and Associated Phenotypes

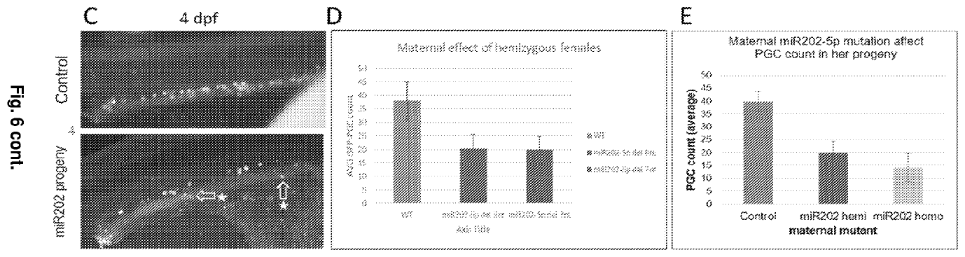

To understand the role of miR202 in Nile tilapia, we used engineered nucleases to abrogate its function. We targeted a site in precursor miR202 gene adjacent to the seed sequence. Alongside the miR202 sequence, we co-target a pigmentation gene (Tyrosinase: tyr) to serve as a mutagenesis selection marker. Twenty embryos with the most severe pigmentation defect were selected and raised to adulthood. We observed that all selected miR202 F0 developed normally. Since miR-202-5p was described as a maternal factor in zebrafish studies [4], we investigated whether PGC development was affected in maternal defect of miR202 mutant embryos. Two F0 mutant females were crossed with wild-type (WT) male tilapia and their embryos progeny was analyzed under fluorescent microscopy to score GFP-PGC count. The number of PGCs in 4 days post fertilization (dpf) embryos was significantly lower than that of embryos from WT mother (FIG. 6B) and ~50% of embryos displayed mislocalized PGCs (FIG. 6A, bottom panel). We found no evidence of PGC development defect in the progeny from F0 mutant male with WT female (FIG. 6B) suggesting no paternal or zygotic effect of miR202 mutations. F1 Progeny from F0 males with WT females were further screened for germline transmission of indel mutations. To this end, F₁ embryos were raised to 3 months of age and genotyped by Sanger sequencing of the PCR amplicon containing the targeted miR202 loci. To establish mutant line for further analysis, we selected F1 fish with mutations that completely or partially remove the miR-202-5p seed sequence with a 7-bp (SEQ ID NO: 163), 8-bp (SEQ ID NO: 164) and 15-bp (SEQ ID NO: 165) deletions (FIG. 7). In some examples, the herein disclosed crossing may be performed by mixing sperm and eggs.

### Example 3—Dosage Sensitivity of Maternal miR-202 in Tilapia Embryogenesis, Controlling PGC Development

We observed that F1 tilapia carrying all miR202 heterozygous mutant alleles developed normally and appeared healthy, differentiating into fertile adults of both sexes. The absence of a reproductive phenotype in these sexually mature F1 generation is unsurprising given the presence of a WT allele in all cells of selected mutant. Given the apparent critical role of miR202 in PGC development, we further tested whether decreasing the maternal dose of functional miR202 decreases the number of PGCs. Indeed, in oocytes of heterozygote mutant females, both alleles are expressed but only one code for a functional miR. Thus, if the miR202 gene works in a dose dependent manner, we should expect the progeny from heterozygote females crossed to WT or mutant males to show reduction in the number of PGC. We found that miR202+/− mutant mothers produce a progeny with a 50% reduction in PGC count irrespective of the embryo genotypes and that of the father (FIG. 6D). This result confirmed that this gene dosage sensitivity is maternal specific with no paternal or zygotic effect. Our findings suggest miR202 is maternally expressed in oocyte, stored as a maternal transcript in unfertilized eggs and act as a critical constituent of the germ plasm in developing embryos.

### Example 4—Tilapia miR202 is Required for Female Reproductive Success

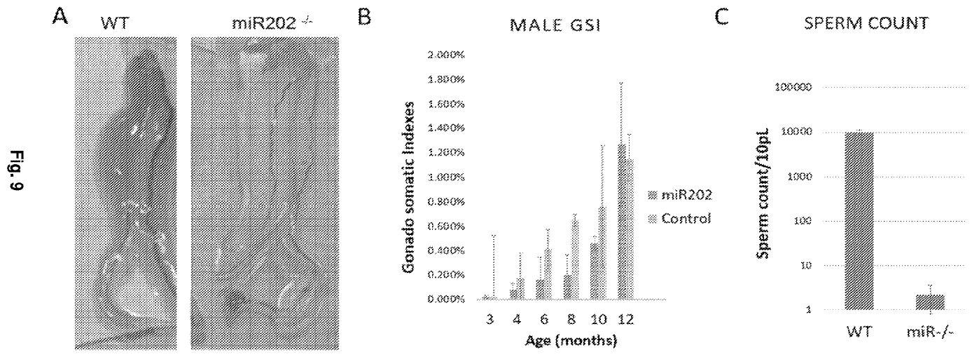

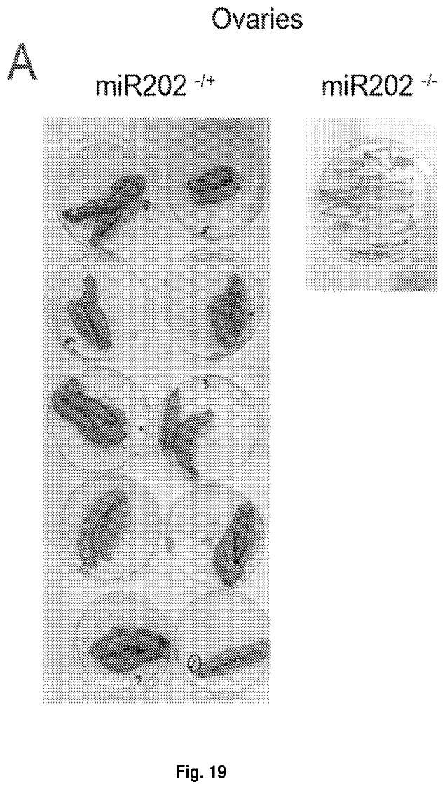

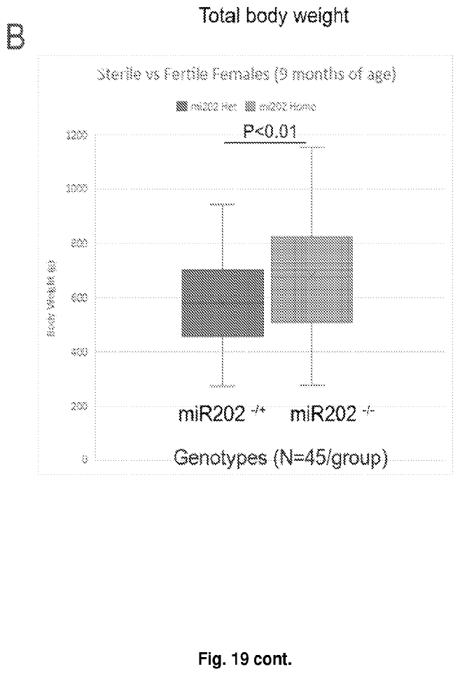

F1 fish carrying the 7-bp, 8-bp and 15-bp deletions were used to generate stable heterozygous F2 line for each allele. Incrossing F2s produced homozygous for each mutation at the expected Mendelian ratio (25%). We observed no external morphological anomalies in any genotype produced except for underdeveloped urogenital papillae in miR202−/− homozygotes (FIG. 5B). We dissected controls (miR202+/+ and miR202+/−) and homozygous mutant females at time intervals and analyzed the gross morphology and histology of their gonads (FIG. 5). When comparing fish with similar length and body weight we observed that mutant ovaries were significantly smaller and thinner than those of the control individuals (FIG. 5A) as evidence by their significant smaller gonadosomatic index (GSI=gonadal weight/total body weight; P<0.05) (FIG. 5D). These immatures, thread like ovaries were observed across all age groups from 3 to 9 months in all female mutant analyzed (N>100) (FIG. 5C). Histological analysis showed that the ovaries from the control fish (miR202+/+ and miR202+/−) contained follicles at all developmental stages including early and late primary growth and full-grown follicles (FIG. 8C). In contrast, the ovaries from miR202−/− fish contained almost exclusively early previtellogenic follicles, suggesting defective regulation of the early steps of folliculogenesis (FIGS. 8A and B). Consistent with these analysis, mutant females placed in aquaria from age 6-9 months failed to spawn while control females showed normal spawning cycles and number of eggs per female starting around 6-7 months of age (one spawn/3-4 weeks). Altogether our results indicate stable and completely penetrant female infertility phenotype caused by follicular development being arrested completely at primary growth stage (previtellogenic stage) until at least 9 months of age. At 9 months of age, we observed that sterile females grew significantly larger than their fertile sibling counterparts (see FIG. 19). In some examples, the herein disclosed incrossing may be performed by mixing sperm and eggs.

### Example 5—Tilapia Mir202 is Required for Male Reproductive Success

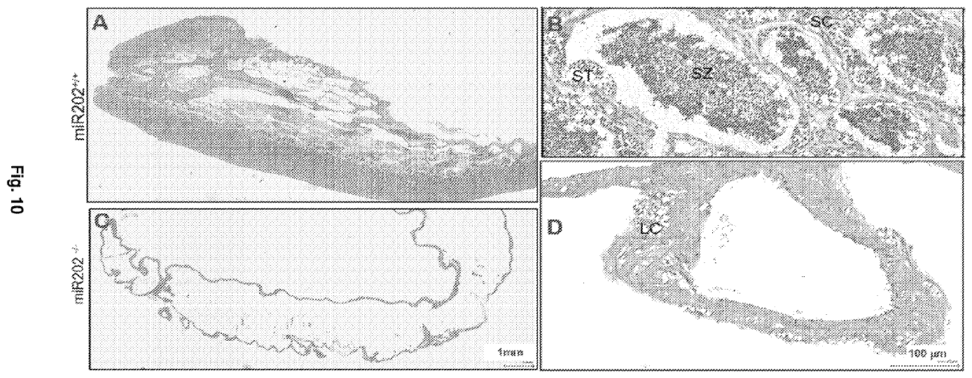

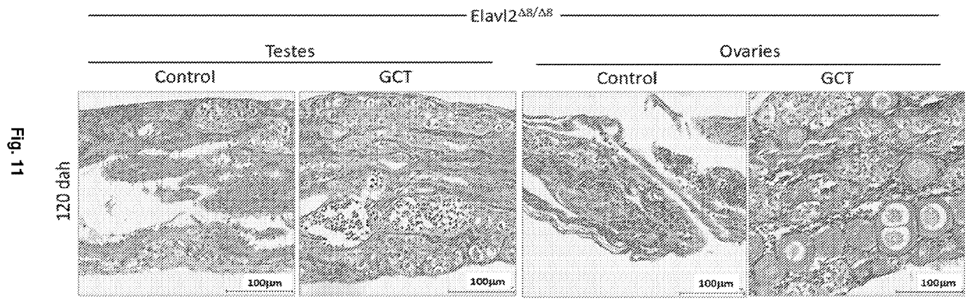

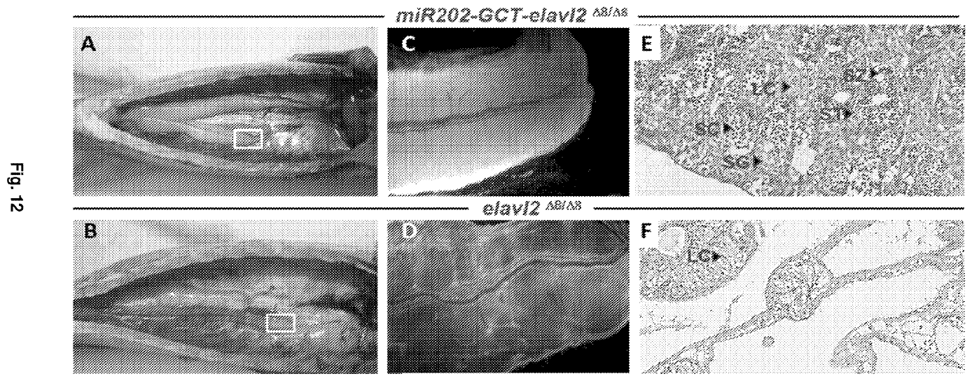

Compared with control (miR202+/+ and miR202+/−), homozygous mutation of miR202 gene led to the development of atrophic male urogenital papillae, as well as smaller and translucent testis (FIG. 9A). We measured a decline of GSI in juvenile miR202−/− (FIG. 9B), however, mutant fish over 6 months of age exhibited varied levels of testicular hydration including hyperhydrated testes in older age groups (9-12 mo) leading to significant GSI increase. Histological analysis showed that the testes from control fish at 6 month of age contained dense populations of spermatogenic cells at all developmental stages, including spermatogonia, spermatocytes and spermatids arranged in cystic structures. Spermatozoa were also found at high density in the lumen of seminiferous tubules in the testis of control males (FIGS. 10A and B). In contrast, the testes from sibling miR202−/− fish revealed patterning disorder and dramatic reduction the total number of spermatogenic cysts (FIGS. 10C and D). Consistent with severe spermatogenesis defect at the morphological and histological level, we measured a sperm count ranging from 0.0001 to 0.001 million cells/ml in seminal fluid from miR202 mutant compared to 1-5 million sperm cells/ml in WT controls (FIG. 9C). We further tested the control and miR202−/− male reproductive success in breeding hapas. In our study we found that 75% of miR202−/− males chosen at random failed to fertilize eggs from wild-type females during repeated mating through natural spawning. The remaining 25% of these males (2 out of 8) managed to produce a small number of viable embryos, achieving an average fertilization success rate of 4% (as detailed in Table 1). The overall average fertilization success rate for all miR202−/− males was 0.7%. In contrast, miR202+/+ sibling males displayed a significantly higher success rate, yielding 85% viable progeny (refer to Table X1). In contrast, miR202+/− sibling males crossed with the same WT females fertilized 85% of eggs produced (Table 2).

TABLE 1

| miR202^{-/-} | | # of eggs | # live embryos at | % of |
|---|---|---|---|---|
| male# | # of Cross | collected | 1-2 dpf | fertilization |
| male_1 | 2 | 1800 | 0 | 0.00% |
| male_2 | 2 | 2000 | 0 | 0.00% |
| male_3 | 2 | 1500 | 0 | 0.00% |
| male_4 | 2 | 3500 | 0 | 0.00% |
| male_5 | 2 | 2000 | 0 | 0.00% |
| male_6 | 2 | 1855 | 55 | 2.96% |
| male_7 | 1 | 487 | 35 | 7.19% |
| male_8 | 1 | 1058 | 0 | 0.00% |
| Total | 14 | 14200 | 90 | 0.63% |
| Summary | | | | |

TABLE 2

| miR202^{+/-} | | # of eggs | # live embryos at | % of |
|---|---|---|---|---|
| male# | # of Cross | collected | 1-2 dpf | fertilization |
| male_1 | 2 | 800 | 780 | 97.50% |
| male_2 | 2 | 2020 | 1800 | 89.11% |
| male_3 | 2 | 1430 | 1240 | 86.71% |
| male_4 | 2 | 2100 | 2000 | 95.24% |
| male_5 | 2 | 3150 | 2300 | 73.02% |
| male_6 | 2 | 900 | 880 | 97.78% |
| male_7 | 2 | 1950 | 1790 | 91.79% |
| male_8 | 2 | 3300 | 1610 | 48.79% |
| Total | 16 | 15650 | 12400 | 84.99% |
| Summary | | | | |

The miR202−/− males under study were individually tagged and placed in natural spawning tanks alongside three wild-type (WT) females aged 12-13 months. Eggs or yolk-stage fry were collected at least twice weekly and counted. Each spawn was identified by tag number, and the count included unfertilized eggs or developing embryos collected. The fertilization rate, calculated as the percentage of fertilized eggs out of the total collected, was determined. Females carrying eggs in their mouths were removed and replaced. After one month, the miR202−/− males were removed from the breeding tank and replaced with fertile miR202+/− sibling control males from the same parental cross (miR202+/− parents). The number of spawns and fertilization success rates were record. In some examples, the herein disclosed crossing may be performed by mixing sperm and eggs.

Altogether our result indicates highly penetrant male spermatogenic disruption associated with male subfertility.

### Example 6—Tilapia Reproductive Capacity is Controlled by miR202 Expression in Somatic Gonadal Cells

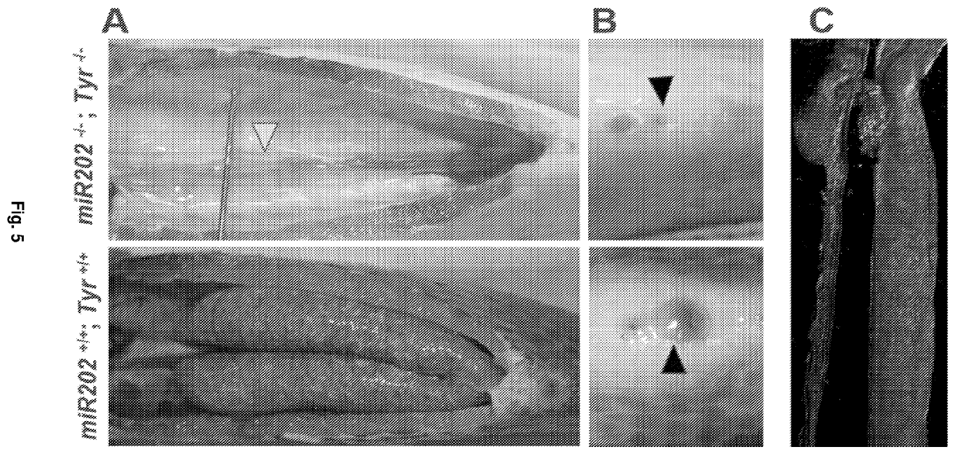

Our results showing that miR202 is a germ plasm-specific RNA involved in PGC survival and migration, indicate that miR202 is maternally expressed in tilapia oocytes. We further established that miR202 play a key regulatory role at different developmental stages of the male and female gonads. Whether the gonadal somatic or the germ cells miR202 expression are causative in determining infertility remains unclear. To analyze the tissue specific contribution of miR202 to germ cell production we created a chimera made of miR202+/+ somatic cell and miR202−/− germ cells. Our approach to generate germ-line replacement chimeras is outlined in FIG. 4. To ensure that recipient fish only produce donor derived gametes we transplanted germ cells extracted from juvenile donor (miR202−/− or WT control) into recipient embryos progeny of dnd1 or Elavl2 heterozygous, and only raised transplanted dnd1−/− [5] or Elavl2−/− [5] and non-transplanted controls to adulthood. The dnd1 and Elavl2 alleles are a fully penetrant recessive zygotic sterile mutation, causing loss of endogenous germ cells (FIGS. 11-13). In these mutants, the gonadal microenvironment is believed to be normal as the genes are exclusively expressed in germ cells. We performed histological analysis of the testes and ovaries from transplanted and control fish at 4 months of age and observed normal spermatogenesis and oogenesis in 66% (N=4/6) and 50% (3/6) of miR202 transplanted recipients while all non-transplanted elavl2−/− were all germ cell-less (FIG. 11). Of all chimeras that survived to adulthood: 20 and 10 were derived from miR202-mutant and WT donors respectively. Of the 20 miR202 chimeras raised, 12 have proven fertile (4 females and 8 males). Of the 10 chimeras derived from WT donor, 9 have proven fertile. The higher colonization efficiency derived from WT donor suggests that higher number of germ line stem cells were present in the gonad of WT fish compared to miR202−/−.

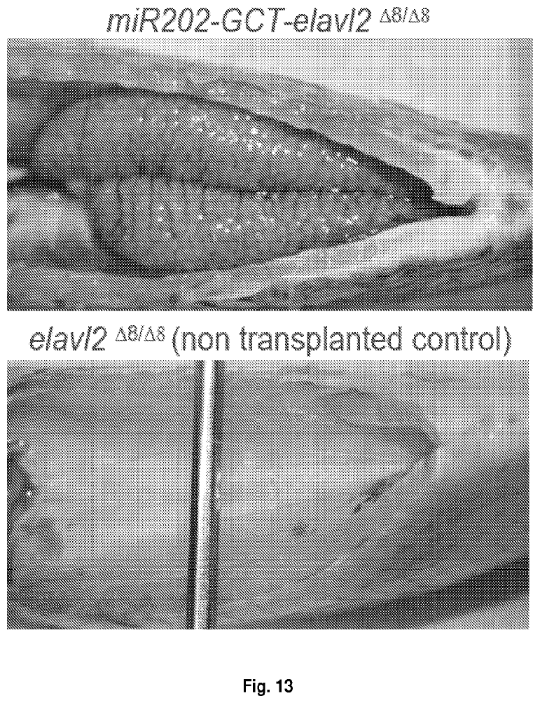

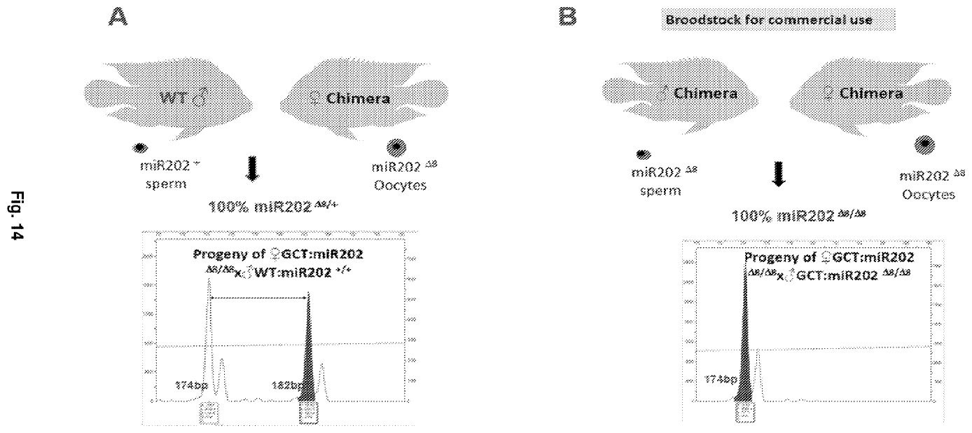

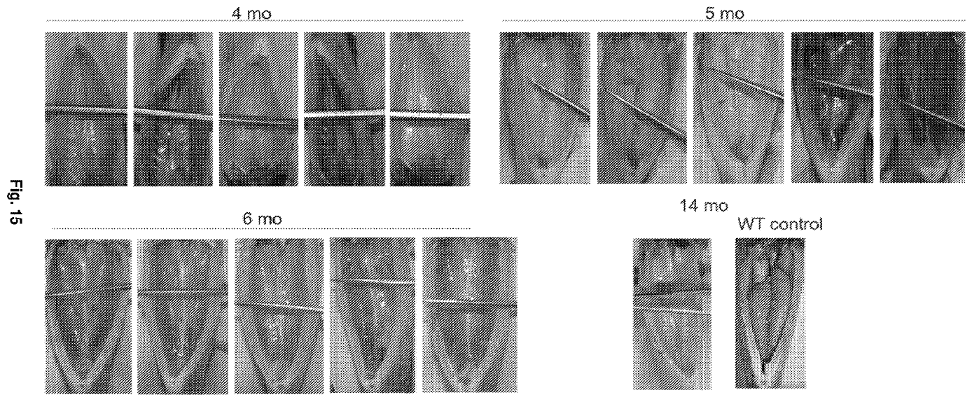

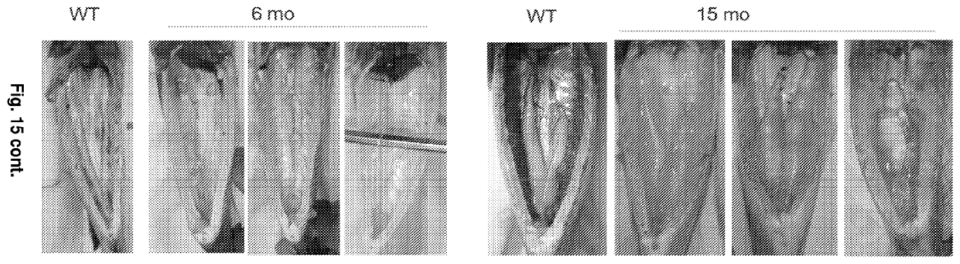

We found that Elavl2 homozygous recipient males that underwent germ cell transplantation (GTP) produce transplanted miR202 mutant sperm at concentration identical to chimeras that had received germ cell transferred from WT donors, suggesting that miR202−/− germ cells have unimpaired proliferation and differentiation capacity. We also found that female chimera developed functional ovaries indicating that miR202 expression in oocyte is dispensable for oogenesis (FIG. 13). In contrast, male and female recipients that did not undergo GTP did not show gonadal development and remained sterile (FIGS. 11-13). Our results indicate successful long-term survival, proliferation, and differentiation of the donor-derived spermatogonia or oogonia into fully functional gametes in the allogenic recipient testes and ovaries.

Altogether these findings indicate that miR202 expression in somatic cells of the gonads (Sertoli and granulosa cells) is necessary for normal gametogenesis. Thus, in tilapia sterility caused by miR202 deficiency does not lie with the germ cell itself, but with the somatic cell types making up the spermatogenic and oogenic microenvironment. We concluded that somatic miR202 is critical for gonadal-somatic cell development or interactions between somatic and germ cells.

### Example 7—Tilapia Reproductive Capacity is Controlled by miR202 Expression in Somatic Gonadal Cells

The complete replacement of the host germ line with donor miR202 germ-line fertile generated maternal mutants for miR202 and provided the tool for studying the full maternal effects of these zygotic sterile mutations. We confirmed that germ cell transplanted (GCT)-female crossed with WT male produce viable embryos. These embryos showed a 60% PGC reduction (avg=16 vs 40 PGC/embryo, FIG. 6E). We incrossed miR202-GCT recipient and genotyped their progeny. We found that all 25 embryos tested are homozygous miR202 mutants (FIG. 14B). We raised progeny from this cross and analyzed their sex and gonadal structures at time intervals. We found that they grew into a mix-sex population of infertile females (FIG. 15A) and infertile males (FIG. 15B) with immature gonads for up to 14 months of age. In some examples, the herein disclosed crossing or incrossing may be performed by mixing sperm and eggs.

### Example 8—miR202-GCT Broodstock can Mass Produce Infertile Progeny

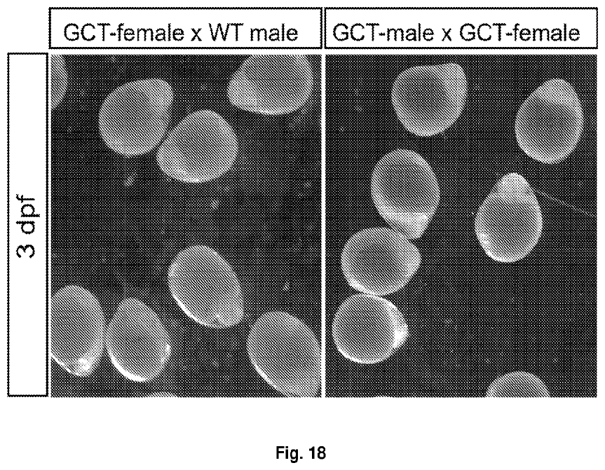

To test whether donor-derived cells had completely replaced the germ lines of the hosts, we transplanted (tyr−/−, miR202−/−) germ cell into (Elavl2−/−) recipients. Chimeric, GCT-females were mated with WT stocks and chimeric GCT-males and the progeny pigmentation was first analyzed. The progeny of GCT-female with WT male consisted only of embryos with a dark melanic pigment color. The progeny of GCT-female×GCT-male consisted of embryos with the completely white phenotype typical of the homozygous tyr−/− albino mutation (FIG. 18). This demonstrates that germ cells from recipient had the Tyr mutant alleles, because completely albino could only result from matings in which both male and female gametes had the Tyr mutation. To further confirm the genotypes of the progeny, we analyzed DNA from 100 embryos progeny resulting from the mating of a female and a male recipient. Of the 3×30 progenies from such intercrosses and incrosses, 3×30 heterozygote (miR202+/−) and 3×30 homozygous miR202−/− mutant were identified. This further demonstrated that both the tyr and miR202 haplotypes were transmitted to the progeny of the recipient. Thus, all GCT-animals obtained were capable only of producing gametes of host origin as our analyses indicate no significant deviation from the expected genotypes for all chimeras tested. We incrossed 3 GCT females individually with at least one GCT male and one WT male and produced a total of 2×5 clutches of half siblings. We genotyped 20 offspring representatives of each half sibling groups by PCR fragments analysis and confirmed them to be 100% and 100% miR202−/−. All batches of progeny showed normal development and differentiated into phenotypic males and females. We systematically observed male bias sex ratio >70% in the miR202−/− progeny (see Table 3). The biased sex ratio in the progeny from GCT female is expected. Indeed, the germ-cell grafts were made using testicular extract (XY germ cells) transplanted into sterile male and female recipient embryos. Hence, half of all recipients involve hosts and donors of different sex with GCT-female developing sex-reversed XY oocytes. Thus, female tilapia with transplanted spermatogonia produced X and Y eggs giving a female to male ratio in the donor-derived progeny of one female to three males (XY×XY=1XX+2XY+1YY supermales).

TABLE 3

| Spermatogonia | Spermatogonia | | | Sex ratio | | | Sex |
|---|---|---|---|---|---|---|---|
| GCT (XY)- | GCG (XY)- | WT (XX) | Fertilization | | | total number | chromosome |
| Male # | Female # | female # | rate (%) | Male (%) | Female (%) | fish sexed | set |
| 1 | 1 | | 87% | 80% (n = 110) | 20% (n = 26) | 136 | XY-XX-YY |
| 2 | 2 | | 100% | 78% (n = 216) | 22% (n = 60) | 276 | XY-XX-YY |
| 3 | 3 | | 76% | 75% (n = 80) | 25% (n = 27) | 107 | XY-XX-YY |
| 3 | | 1 | 85% | 51% (n = 34) | 49% (n = 33) | 67 | XY-XX |
| 2 | | 1 | 87% | 54% (n = 74) | 45% (n = 62) | 136 | XY-XX |
| 1 | | 2 | 92% | 53% (n = 76) | 47% (n = 68) | 144 | XY-XX |

Table 3 displays the sex ratio resulting from germ cell-transplanted male and female tilapia that received spermatogonia (XY), as well as control crosses between GCT males and WT (XX) females. Phenotypic sex was determined at 3 months of age through morphological assessment of the urogenital papillae and dissection of the gonads. In some examples, the herein disclosed crossing or incrossing may be performed by mixing sperm and eggs.

### Example 9—Surrogate Broodstock can Generate Progeny Populations Consisting Predominantly of Either Males or Females, Depending on the Sex of the Donor

All batches of offspring exhibited normal development, differentiating into phenotypic males and females, with sex ratios strongly biased towards either males (>73%) or females (>95%) (see Table 3 and Table 4, respectively). This bias depended on whether the transplanted germ cells were oogonia or spermatogonia. Such biased sex ratios were anticipated since the germ-cell grafts were created using testicular extract (XY germ cells) or ovarian extract (XX germ cells), transplanted into sterile male and female recipient hatchlings. Consequently, progeny resulting from oogonia-injected recipients possessed only X chromosomes and all developed into XX females, with frequencies ranging from 92 to 100% (see Table 4). Conversely, females and males tilapia transplanted with spermatogonia produced both X and Y eggs (pseudo-females) and sperm, leading to a female-to-male ratio in the donor-derived progeny of one female to three males (XY×XY=1XX+2XY+1YY supermales) (see Table 3).

Identification of YY supermales and their contribution to an all-male progeny was achieved as follows: Six males from the progeny of a cross between a pseudo-female (XY) and a genetic male (XY) were randomly selected and paired with regular wild-type females. Among the resulting offspring, two F2 males (#2 and 4, Table 5) produced 100% and 96% male F3 progeny, respectively. In contrast, the remaining four F2 males produced F3 progeny with male-to-female ratios ranging from 47% to 67%, akin to the control group (see Table 5). Therefore, two out of the six F2 males (30.0%) were identified as YY supermales.

To verify the reliability of YY supermales, two additional populations were established by mating them with females from different farms or the same family that produced YY supermales. Offspring from females sourced from other farms exhibited an impressive male rate of 99.5% among 200 individuals (n=200). Similarly, offspring from females of the same family as YY supermales showed a male rate of 94.1% among 187 individuals (n=187). These findings underscore the suitability of the offspring generated in this study for tilapia aquaculture.

TABLE 4

| Oogonia | Oogonia | | | Sex ratio | | | Sex |
|---|---|---|---|---|---|---|---|
| GCT (XX) - | GCG (XX) - | WT (XY) | Fertilization | | | total number | chromosome |
| Male # | Female # | male # | rate (%) | Male (%) | Female (%) | fish sexed | set |
| 1 | 1 | | 95% | 4% (n = 4) | 96% (n = 95) | 99 | XX |
| 2 | 2 | | 100% | 0% (n = 0) | 100% (n = 100) | 100 | XX |
| 3 | 3 | | 94% | 8% (n = 10) | 92% (n = 95) | 105 | XX |
| | 3 | 1 | 89% | 41% (n = 43) | 59% (n = 62) | 105 | YX-XX |
| | 2 | 2 | 100% | 47% (n = 49) | 53% (n = 55) | 104 | XY-XX |
| | 1 | 3 | 93% | 52% (n = 53) | 48% (n = 49) | 102 | XY-XX |

Table 4 displays the sex ratio resulting from germ cell-transplanted male and female tilapia that received oogonia (XY), as well as control crosses between GCT female and WT (XY) males. Phenotypic sex was determined at 3 months of age through morphological assessment of the urogenital papillae and dissection of the gonads.

TABLE 5

| | | | Progeny Sex ratio | | | Sex |
|---|---|---|---|---|---|---|
| Male # | WT Female | Fertilization | | | total number | chromosome |
| (XY or YY) | (XX) | rate (%) | Male (%) | Female (%) | fish sexed | set |
| 1 | 1 | 95% | 64% (n = 65) | 36% (n = 37) | 102 | XY |
| 2 | 1 | 100% | 100% (n = 96) | 0% | 96 | YY |
| 3 | 1 | 94% | 55% (n = 55) | 45% (n = 45) | 100 | XY |
| 4 | 1 | 100% | 96% (n = 103) | 4% (n = 4) | 107 | YY |
| 5 | 1 | 100% | 57% (n = 59) | 43% (n = 43) | 103 | XY |
| 6 | 1 | 93% | 48% (n = 49) | 52% (n = 53) | 102 | XY |

Table 5 presents the fertilization rate, as well as the percentage of males and females obtained from potential YY males of Nile tilapia (derived from a cross as described in Table Y1) paired with a WT female. Phenotypic sex was determined at 3 months of age through morphological assessment of the urogenital papillae and dissection of the gonads. In some examples, the herein disclosed crossing may be performed by mixing sperm and eggs.

### Example 10: Sterile Female (miR202−/−) Fish Surpassed Fertile Controls (miR202+/−) in Performance

We observed no weight difference between miR202+/− and miR202−/− females up to 6 months of age. However, over the following 3 months, we noted a gradual improvement in growth rate. By 9 months of age, homozygous mutant females exhibited a significant 17% increase in average body weight compared to the control miR202+/− group. Unsurprisingly, 9 months old, females with the miR202−/− mutation had gonads arrested at the previtellogenic stage, while the control group's gonads were fully mature. Thus, the miR202−/− mutation in females had no discernible positive or negative impact on overall fish performance up to 6 months of age, when females typically begin to sexually mature. Interestingly, we observed that tilapia females with atrophic gonads gradually outperformed their fully fertile counterparts. This finding supports the idea that energy not allocated to ovarian development can be redirected to somatic growth.

### Example 11—Genetic Balancing Strategy to Maintain the Lineage of Elavl2 Mutation in Heterozygotes

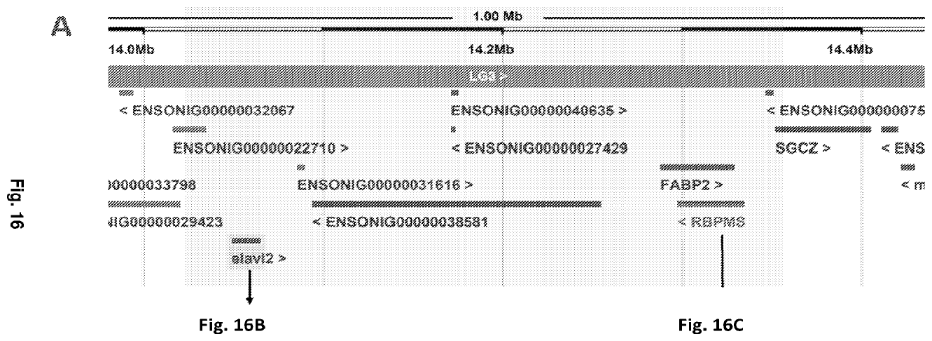

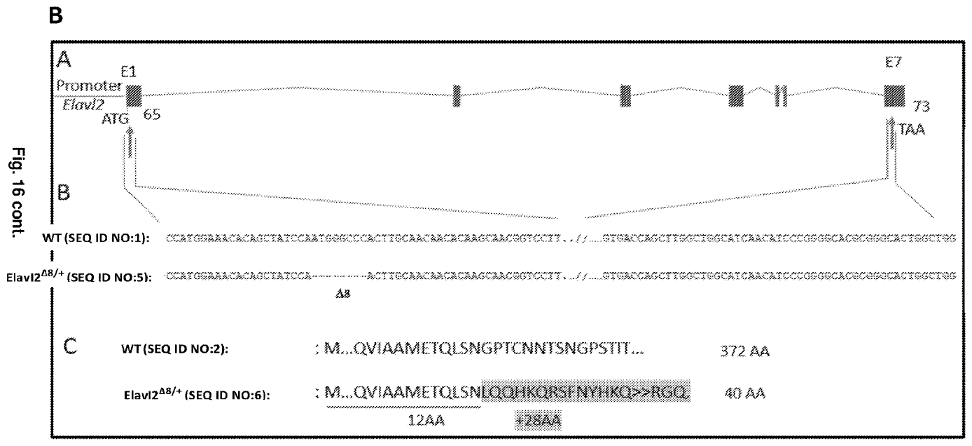

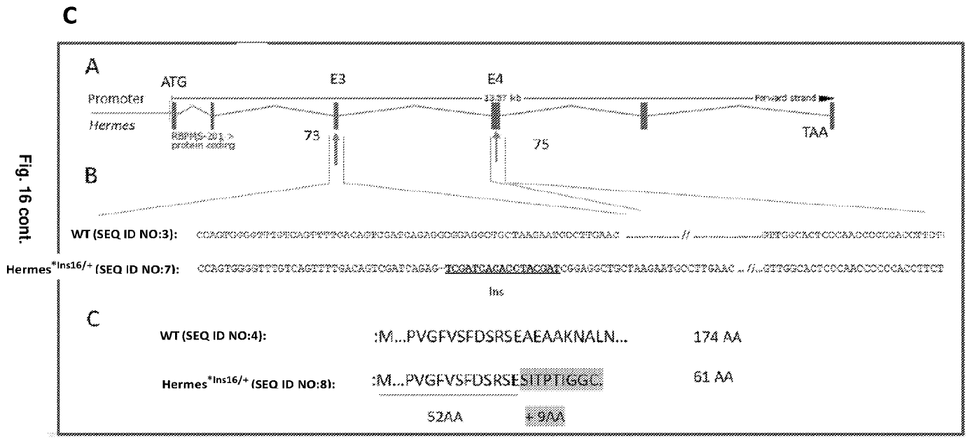

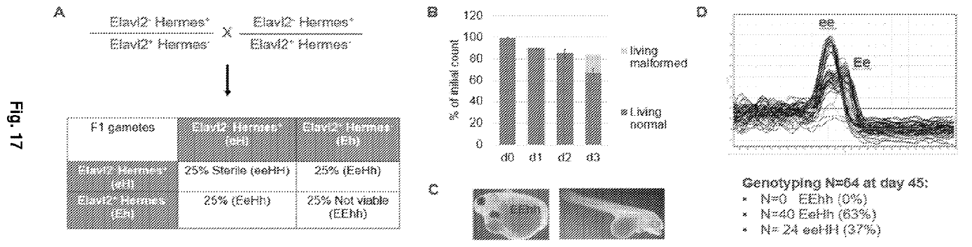

In earlier studies we demonstrated that both Elavl2 and dnd1 heterozygous mutant parents can produce 25% of germ-cell-free homozygous recipient hatchlings for intraperitoneal transplantation of germ cell extracts (see PCT Publication No. WO2020/033940, which is incorporated by reference). To ensure maintenance of Elavl2 hemizygote individuals and further increase the pool of acceptable hatchling recipient for germ cell transplantation we created a recessive lethal mutation in another gene adjacent to Elavl2 on the homologous chromosome. We located Hermes (Rbmps) in close proximity to Elavl2 (200 kb ~0.2 centimorgan) (FIG. 16A) and generated a mutant allele of Hermes with a 16-nucleotide insertion predicted by conceptual translation to produce a truncated Hermes protein (FIG. 16B). We found that the frameshift mutant Hermes^{Ins16/Ins16} line is non-viable. We then bred a female Hermes^{Ins16/+} with a male Elavl2^{Δ8/+}, selected and raised double heterozygote mutant Hermes^{Ins16/+}, Elavl2^{Δ8/+} to adulthood (FIG. 17A). Finally, we incrossed double heterozygous mutant and raised 200 of their progenies for further analysis. We found that ~20% of the progeny showed malformities at 3-4 dpf. We genotyped the progeny and found that ~75% of the Hermes homozygous mutant died within 7 days post fertilization (FIG. 17B). Nine days post fertilization, the remaining Hermes^{Ins16/Ins16} escapes showed cranio-facial and body axis deformities (FIG. 17C). We scored no viable Hermes^{Ins16/ns16} among the progeny segregated from a heterozygote at one month of age, confirming that Hermes^{Ins16/Ins16} mutant are non-viable (FIG. 17D). We further used this progeny population for germ cell transplantation experiments. Genotyping of germ cell transplanted hatchlings at 45 days post transplantation resulted in ~37% of the treated fish being Elavl2^{538 8/538 8}, while the remaining were double heterozygote (Hermes^{Ins16/+}, Elavl2^{D8/+} or eEhH). These results confirm that a lethal balancing mutation can be used to eliminate the Elavl2^{+/+} zygotes from the pool of progeny. Our results almost perfectly match the expected Mendelian distribution of genotype from completely linked genes (33% and 66%). Furthermore, given that germ-cell-free Elavl2-knockout are the only suitable recipients for germ cell transplantation, this strategy provides the added benefit of increasing by ~30% the proportion of recipient sterile fish needed for that will only produce donor derived germ cells. In some examples, the herein disclosed incrossing may be performed by mixing sperm and eggs.

### REFERENCES

1. Tait-Burkard, C., et al., Livestock 2.0-genome editing for fitter, healthier, and more productive farmed animals. Genome biology, 2018. 19(1): p. 1-11.
2. Modzelewski, A. J., et al., Dgcr8 and Dicer are essential for sex chromosome integrity during meiosis in males. Journal of cell science, 2015. 128(12): p. 2314-2327.
3. Dong, Z., et al., Generation of myostatin B knockout yellow catfish (Tachysurus fulvidraco) using transcription activator-like effector nucleases. Zebrafish, 2014. 11(3): p. 265-274.
4. Zhang, J., et al., MiR-202-5p is a novel germ plasm-specific microRNA in zebrafish. Scientific reports, 2017. 7(1): p. 1-7.
5. PCT Publication No. WO2020/033940.

In the preceding description, for purposes of explanation, numerous details are set forth in order to provide a thorough understanding of the embodiments. However, it will be apparent to one skilled in the art that these specific details are not required.

The above-described embodiments are intended to be examples only. Alterations, modifications and variations can be effected to the particular embodiments by those of skill in the art. The scope of the claims should not be limited by the particular embodiments set forth herein, but should be construed in a manner consistent with the specification as a whole.

What is claimed is:

1. An endogenous germ cell-less fish, crustacean, or mollusk having a chimeric gonad, wherein the chimeric gonad comprises at least one transplanted germ cell having a mutation that is present in the germline and disrupts the development and/or function of somatic gonadal cells.

2. The endogenous germ cell-less fish, crustacean, or mollusk of claim 1, wherein the endogenous germ cell-less fish, crustacean, or mollusk is homozygous wild type in the germline at the locus for the mutation that is present in the germline and disrupts the development and/or function of somatic gonadal cells.

3. The endogenous germ cell-less fish, crustacean, or mollusk of claim 1 or 2, wherein disrupting the development and/or function of somatic gonadal cells comprises disrupting gametogenesis.

4. The endogenous germ cell-less fish, crustacean, or mollusk of any one of claims 1-3, wherein disrupting the development and/or function of somatic gonadal cells comprises disrupting the development and/or function of testis and/or ovary cells.

5. The endogenous germ cell-less fish, crustacean, or mollusk of any one of claims 1-4, wherein disrupting the development and/or function of somatic gonadal cells comprises disrupting the development and/or function of Sertoli, Theca, Granulosa, and/or Leydig cells.

6. The endogenous germ cell-less fish, crustacean, or mollusk of any one of claims 1-5, wherein disrupting the development and/or function of somatic gonadal cells comprises disrupting at least one signaling molecule produced by Sertoli, Theca, Granulosa, and/or Leydig cells.

7. The endogenous germ cell-less fish, crustacean, or mollusk of any one of claims 1-6, wherein disrupting the development and/or function of somatic gonadal cells comprises disrupting at least one secreted diffusible signal protein or growth factor protein that regulates Spermatogonial stem cell (SCC) and/or Oogonial stem cell (OSC) renewal and/or differentiation.

8. The endogenous germ cell-less fish, crustacean, or mollusk of claim 7, wherein the at least one secreted diffusible signal protein or growth factor protein is glial cell line-derived neurotrophic factor (GDNF); bone morphogenetic protein 4 (BMP4); stem cell factor (SCF); fibroblast growth factor 2 (FGF2); C-X-C motif chemokine 12 (CXCL12); and/or epidermal growth factor (EGF).

9. The endogenous germ cell-less fish, crustacean, or mollusk of any one of claims 1-8, wherein the mutation that is present in the germline and disrupts the development and/or function of somatic gonadal cells is a mutation in microRNA miR-202.

10. The endogenous germ cell-less fish, crustacean, or mollusk of any one of claims 1-9, wherein the at least one germ cell is from about 500 germ cells to about 6,000 germ cells.

11. The endogenous germ cell-less fish, crustacean, or mollusk of any one of claims 1-10, wherein the at least one germ cell is a Spermatogonial stem cell (SCC) or an Oogonial stem cell (OSC).

12. The endogenous germ cell-less fish, crustacean, or mollusk of any one of claims 1-11, wherein the at least one germ cell is from a heterogametic male donor, such as XY.

13. The endogenous germ cell-less fish, crustacean, or mollusk of any one of claims 1-11, wherein the at least one germ cell is from a homogametic female donor, such as XX.

14. The endogenous germ cell-less fish, crustacean, or mollusk of any one of claims 1-11, wherein the at least one germ cell transplanted into the endogenous germ cell-less fish, crustacean, or mollusk that is female; and the endogenous germ cell-less fish, crustacean, or mollusk that is male, is an Oogonial stem cell (OSC) from a homogametic female donor, such as XX.

15. The endogenous germ cell-less fish, crustacean, or mollusk of any one of claims 1-11, wherein the at least one germ cell transplanted into the endogenous germ cell-less fish, crustacean, or mollusk that is female; and the endogenous germ cell-less fish, crustacean, or mollusk that is male, is a Spermatogonial stem cell (SCC) from a heterogametic male donor, such as XY.

16. The endogenous germ cell-less fish, crustacean, or mollusk of any one of claims 1-11, wherein the at least one germ cell is from a homogametic male donor, such as ZZ.

17. The endogenous germ cell-less fish, crustacean, or mollusk of any one of claims 1-11, wherein the at least one germ cell is from a heterogametic female donor, such as WZ.

18. The endogenous germ cell-less fish, crustacean, or mollusk of any one of claims 1-11, wherein the at least one germ cell transplanted into the endogenous germ cell-less fish, crustacean, or mollusk that is female; and the endogenous germ cell-less fish, crustacean, or mollusk that is male, is a Oogonial stem cell (OSC) from a heterogametic female donor, such as WZ.

19. The endogenous germ cell-less fish, crustacean, or mollusk of any one of claims 1-11, wherein the at least one germ cell transplanted into the: endogenous germ cell-less fish, crustacean, or mollusk that is female; and the endogenous germ cell-less fish, crustacean, or mollusk that is male, is a Spermatogonial stem cell (SCC) from a homogametic male donor, such as ZZ.

20. The endogenous germ cell-less fish, crustacean, or mollusk of any one of claims 1-11, wherein the at least one germ cell transplanted into the endogenous germ cell-less fish, crustacean, or mollusk that is female; and the endogenous germ cell-less fish, crustacean, or mollusk that is male, is a Oogonial stem cell (OSC) from a homogametic superfemale donor, such as WW.

21. The endogenous germ cell-less fish, crustacean, or mollusk of any one of claims 1-11, wherein the at least one germ cell transplanted into the: endogenous germ cell-less fish, crustacean, or mollusk that is female; and the endogenous germ cell-less fish, crustacean, or mollusk that is male, is a Spermatogonial stem cell (SCC) from a homogametic supermale donor, such as YY.

22. The endogenous germ cell-less fish, crustacean, or mollusk of any one of claims 1-21, wherein the at least one germ cell is transplanted into the peritoneal cavity of the embryo or hatchling of the endogenous germ cell-less fish, crustacean, or mollusk.

23. The endogenous germ cell-less fish, crustacean, or mollusk of any one of claims 1-22, wherein the endogenous germ cell-less fish, crustacean, or mollusk has a null mutation in dnd1, Elavl2, vasa, nanos3, and/or piwi-like gene.

24. The endogenous germ cell-less fish, crustacean, or mollusk of any one of claims 1-22, wherein the endogenous germ cell-less fish, crustacean, or mollusk is created using ploidy manipulation, such as triploidy manipulation.

25. The endogenous germ cell-less fish, crustacean, or mollusk of any one of claims 1-22, wherein the endogenous germ cell-less fish, crustacean, or mollusk is created by hybridization.

26. The endogenous germ cell-less fish, crustacean, or mollusk of any one of claims 1-22, wherein the endogenous germ cell-less fish, crustacean, or mollusk is created using exposure to high levels of sex hormones.

27. The endogenous germ cell-less fish, crustacean, or mollusk of any one of claims 1-22, wherein the endogenous germ cell-less fish, crustacean, or mollusk is created using morpholinos.

28. The endogenous germ cell-less fish, crustacean, or mollusk of any one of claims 1-22, wherein the endogenous germ cell-less fish, crustacean, or mollusk is created using sterile hybrids.

29. The endogenous germ cell-less fish, crustacean, or mollusk of any one of claims 1-22, wherein the endogenous germ cell-less fish, crustacean, or mollusk is created using chemical exposure.

30. The endogenous germ cell-less fish, crustacean, or mollusk of any one of claims 1-29, wherein the fish, crustacean, or mollusk is Atlantic salmon, Rainbow Trout, Coho Salmon, tilapia, cobia, Seriola spp., Grouper, Snapper, barramundi, Sea Bream, Sea Bass, lumpfish, sturgeon, Litopeneus vannamei, Peneaus monadon, oysters, clams or mussels.

31. The endogenous germ cell-less fish, crustacean, or mollusk of any one of claims 1-24, wherein the fish, crustacean, or mollusk is tilapia.

32. A method of generating an endogenous germ cell-less fish, crustacean, or mollusk, comprising the steps of:
transplanting at least one germ cell having a mutation that is present in the germline and disrupts the development and/or function of somatic gonadal cells, into an endogenous germ cell-less fish, crustacean, or mollusk, producing a chimeric gonad.

33. The method of claim 32, wherein the recipient fish, crustacean, or mollusk is homozygous wild type in the germline at the locus for the mutation that is present in the germline and disrupts the development and/or function of somatic gonadal cells.

34. The method of claim 32 or 33, wherein disrupting the development and/or function of somatic gonadal cells comprises disrupting gametogenesis.

35. The method of any one of claims 32-34, wherein disrupting the development and/or function of somatic gonadal cells comprises disrupting the development and/or function of testis and/or ovary cells.

36. The method of any one of claims 32-35, wherein disrupting the development and/or function of somatic gonadal cells comprises disrupting the development and/or function of Sertoli, Theca, Granulosa, and/or Leydig cells.

37. The method of any one of claims 32-36, wherein disrupting the development and/or function of somatic gonadal cells comprises disrupting at least one signaling molecule produced by Sertoli, Theca, Granulosa, and/or Leydig cells.

38. The method of any one of claims 32-37, wherein disrupting the development and/or function of somatic gonadal cells comprises disrupting at least one secreted diffusible signal protein or growth factor protein that regulates Spermatogonial stem cell (SCC) and/or Oogonial stem cell (OSC) renewal and/or differentiation.

39. The method of claim 38, wherein the at least one secreted diffusible signal protein or growth factor protein is glial cell line-derived neurotrophic factor (GDNF); bone morphogenetic protein 4 (BMP4); stem cell factor (SCF); fibroblast growth factor 2 (FGF2); C-X-C motif chemokine 12 (CXCL12); and/or epidermal growth factor (EGF).

40. The method of any one of claims 32-39, wherein the mutation that is present in the germline and disrupts the development and/or function of somatic gonadal cells is a mutation in microRNA miR-202.

41. The method of any one of claims 32-40, wherein the at least one germ cell is from about 500 germ cells to about 6,000 germ cells.

42. The method of any one of claims 32-41, wherein the at least one germ cell is a Spermatogonial stem cell (SCC) or an Oogonial stem cell (OSC).

43. The method of any one of claims 32-42, wherein the at least one germ cell is from a heterogametic male donor, such as XY.

44. The method of any one of claims 32-42, wherein the at least one germ cell is from a homogametic female donor, such as XX.

45. The method of any one of claims 32-42, wherein the at least one germ cell transplanted into the endogenous germ cell-less fish, crustacean, or mollusk that is female; and the endogenous germ cell-less fish, crustacean, or mollusk that is male, is an Oogonial stem cell (OSC) from a homogametic female donor, such as XX.

46. The method of any one of claims 32-42, wherein the at least one germ cell transplanted into the endogenous germ cell-less fish, crustacean, or mollusk that is female; and the endogenous germ cell-less fish, crustacean, or mollusk that is male, is a Spermatogonial stem cell (SCC) from a heterogametic male donor, such as XY.

47. The method of any one of claims 32-42, wherein the at least one germ cell is from a homogametic male donor, such as ZZ.

48. The method of any one of claims 32-42, wherein the at least one germ cell is from a heterogametic female donor, such as WZ.

49. The method of any one of claims 32-42, wherein the at least one germ cell transplanted into the endogenous germ cell-less fish, crustacean, or mollusk that is female; and the endogenous germ cell-less fish, crustacean, or mollusk that is male, is a Oogonial stem cell (OSC) from a heterogametic female donor, such as WZ.

50. The method of any one of claims 32-42, wherein the at least one germ cell transplanted into the: endogenous germ cell-less fish, crustacean, or mollusk that is female; and the endogenous germ cell-less fish, crustacean, or mollusk that is male, is a Spermatogonial stem cell (SCC) from a homogametic male donor, such as ZZ.

51. The method of any one of claims 32-42, wherein the at least one germ cell transplanted into the endogenous germ cell-less fish, crustacean, or mollusk that is female; and the endogenous germ cell-less fish, crustacean, or mollusk that is male, is a Oogonial stem cell (OSC) from a homogametic superfemale donor, such as WW.

52. The method of any one of claims 32-42, wherein the at least one germ cell transplanted into the: endogenous germ cell-less fish, crustacean, or mollusk that is female; and the endogenous germ cell-less fish, crustacean, or mollusk that is male, is a Spermatogonial stem cell (SCC) from a h homogametic supermale donor, such as YY.

53. The method of any one of claims 32-52, wherein the at least one germ cell is transplanted into the peritoneal cavity of the embryo or hatchling of the endogenous germ cell-less fish, crustacean, or mollusk.

54. The method of any one of claims 32-53, wherein the endogenous germ cell-less fish, crustacean, or mollusk has a null mutation in dnd1, Elavl2, vasa, nanos3, and/or piwi-like gene.

55. The method of any one of claims 32-53, wherein the endogenous germ cell-less fish, crustacean, or mollusk is created using ploidy manipulation, such as triploidy manipulation.

56. The method of any one of claims 32-53, wherein the endogenous germ cell-less fish, crustacean, or mollusk is created by hybridization.

57. The method of any one of claims 32-53, wherein the endogenous germ cell-less fish, crustacean, or mollusk is created using exposure to high levels of sex hormones.

58. The method of any one of claims 32-53, wherein the endogenous germ cell-less fish, crustacean, or mollusk is created using morpholinos.

59. The method of any one of claims 32-53, wherein the endogenous germ cell-less fish, crustacean, or mollusk is created using sterile hybrids.

60. The method of any one of claims 32-53, wherein the endogenous germ cell-less fish, crustacean, or mollusk is created using chemical exposure.

61. The method of any one of claims 32-60, wherein the fish, crustacean, or mollusk is Atlantic salmon, Rainbow Trout, Coho Salmon, tilapia, cobia, Seriola spp., Grouper, Snapper, barramundi, Sea Bream, Sea Bass, lumpfish, sturgeon, Litopeneus vannamei, Peneaus monadon, oysters, clams or mussels.

62. The method of any one of claims 32-61, wherein the fish, crustacean, or mollusk is tilapia.

63. The fish, crustacean, or mollusk produced by the method of any one of claims 32-62.

64. A method of generating a sterile fish, crustacean, or mollusk, comprising the steps of:
breeding (i) an endogenous germ cell-less fish, crustacean, or mollusk of any one of claims 1-31 and 63 that is female with (ii) an endogenous germ cell-less fish, crustacean, or mollusk of any one of claims 1-31 and 63 that is male, to produce the sterile fish crustacean, or mollusk.

65. A method of generating a sterile fish, crustacean, or mollusk, comprising the steps of:
breeding (i) an endogenous germ cell-less fish, crustacean, or mollusk produced by the method of any one of claims 32-62 that is female with (ii) an endogenous germ cell-less fish, crustacean, or mollusk produced by the method of any one of claims 32-62 that is male, to produce the sterile fish crustacean, or mollusk.

66. An endogenous germ cell-less fish, crustacean, or mollusk having a chimeric gonad, wherein the chimeric gonad comprises at least one transplanted: a) Oogonial stem cell (OSC) from a homogametic female donor, such as XX, having a mutation that is present in the germline and disrupts the development and/or function of somatic gonadal cells; or b) Spermatogonial stem cell (SCC) from a homogametic male donor, such as ZZ, having a mutation that is present in the germline and disrupts the development and/or function of somatic gonadal cells.

67. The endogenous germ cell-less fish, crustacean, or mollusk of claim 66, wherein the endogenous germ cell-less fish, crustacean, or mollusk is homozygous wild type in the germline at the locus for the mutation that is present in the germline and disrupts the development and/or function of somatic gonadal cells.

68. The endogenous germ cell-less fish, crustacean, or mollusk of claim 66 or 67, wherein disrupting the development and/or function of somatic gonadal cells comprises disrupting gametogenesis.

69. The endogenous germ cell-less fish, crustacean, or mollusk of any one of claims 66-68, wherein disrupting the development and/or function of somatic gonadal cells comprises disrupting the development and/or function of testis and/or ovary cells.

70. The endogenous germ cell-less fish, crustacean, or mollusk of any one of claims 66-69, wherein disrupting the development and/or function of somatic gonadal cells comprises disrupting the development and/or function of Sertoli, Theca, Granulosa, and/or Leydig cells.

71. The endogenous germ cell-less fish, crustacean, or mollusk of any one of claims 66-70, wherein disrupting the development and/or function of somatic gonadal cells comprises disrupting at least one signaling molecule produced by Sertoli, Theca, Granulosa, and/or Leydig cells.

72. The endogenous germ cell-less fish, crustacean, or mollusk of any one of claims 66-71, wherein disrupting the development and/or function of somatic gonadal cells comprises disrupting at least one secreted diffusible signal protein or growth factor protein that regulates Spermatogonial stem cell (SCC) and/or Oogonial stem cell (OSC) renewal and/or differentiation.

73. The endogenous germ cell-less fish, crustacean, or mollusk of claim 72, wherein the at least one secreted diffusible signal protein or growth factor protein is glial cell line-derived neurotrophic factor (GDNF); bone morphogenetic protein 4 (BMP4); stem cell factor (SCF); fibroblast growth factor 2 (FGF2); C-X-C motif chemokine 12 (CXCL12); and/or epidermal growth factor (EGF).

74. The endogenous germ cell-less fish, crustacean, or mollusk of any one of claims 66-73, wherein the mutation that is present in the germline and disrupts the development and/or function of somatic gonadal cells is a mutation in microRNA miR-202.

75. The endogenous germ cell-less fish, crustacean, or mollusk of any one of claims 66-74, wherein the at least one germ cell is from about 500 germ cells to about 6,000 germ cells.

76. The endogenous germ cell-less fish, crustacean, or mollusk of any one of claims 66-75, wherein the at least one germ cell is transplanted into the peritoneal cavity of the embryo or hatchling of the endogenous germ cell-less fish, crustacean, or mollusk.

77. The endogenous germ cell-less fish, crustacean, or mollusk of any one of claims 66-76, wherein the endogenous germ cell-less fish, crustacean, or mollusk has a null mutation in dnd1, Elavl2, vasa, nanos3, and/or piwi-like gene.

78. The endogenous germ cell-less fish, crustacean, or mollusk of any one of claims 66-76, wherein the endogenous germ cell-less fish, crustacean, or mollusk is created using ploidy manipulation, such as triploidy manipulation.

79. The endogenous germ cell-less fish, crustacean, or mollusk of any one of claims 66-76, wherein the endogenous germ cell-less fish, crustacean, or mollusk is created by hybridization.

80. The endogenous germ cell-less fish, crustacean, or mollusk of any one of claims 66-76, wherein the endogenous germ cell-less fish, crustacean, or mollusk is created using exposure to high levels of sex hormones.

81. The endogenous germ cell-less fish, crustacean, or mollusk of any one of claims 66-76, wherein the endogenous germ cell-less fish, crustacean, or mollusk is created using morpholinos.

82. The endogenous germ cell-less fish, crustacean, or mollusk of any one of claims 66-76, wherein the endogenous germ cell-less fish, crustacean, or mollusk is created using sterile hybrids.

83. The endogenous germ cell-less fish, crustacean, or mollusk of any one of claims 66-76, wherein the endogenous germ cell-less fish, crustacean, or mollusk is created using chemical exposure.

84. The endogenous germ cell-less fish, crustacean, or mollusk of any one of claims 66-83, wherein the fish, crustacean, or mollusk is Atlantic salmon, Rainbow Trout, Coho Salmon, tilapia, cobia, Seriola spp., Grouper, Snapper, barramundi, Sea Bream, Sea Bass, lumpfish, sturgeon, Litopeneus vannamei, Peneaus monadon, oysters, clams or mussels.

85. The endogenous germ cell-less fish, crustacean, or mollusk of any one of claims 66-84, wherein the fish, crustacean, or mollusk is tilapia.

86. A method of generating an endogenous germ cell-less fish, crustacean, or mollusk, comprising the steps of:
transplanting at least one: a) Oogonial stem cell (OSC) from a homogametic female donor, such as XX, having a mutation that is present in the germline and disrupts the development and/or function of somatic gonadal cells; or b) Spermatogonial stem cell (SCC) from a homogametic male donor, such as ZZ, having a mutation that is present in the germline and disrupts the development and/or function of somatic gonadal cells, into an endogenous germ cell-less fish, crustacean, or mollusk, producing a chimeric gonad.

87. The method of claim 86, wherein the recipient fish, crustacean, or mollusk is homozygous wild type in the germline at the locus for the mutation that is present in the germline and disrupts the development and/or function of somatic gonadal cells.

88. The method of claim 86 or 87, wherein disrupting the development and/or function of somatic gonadal cells comprises disrupting gametogenesis.

89. The method of any one of claims 86-88, wherein disrupting the development and/or function of somatic gonadal cells comprises disrupting the development and/or function of testis and/or ovary cells.

90. The method of any one of claims 86-89, wherein disrupting the development and/or function of somatic gonadal cells comprises disrupting the development and/or function of Sertoli, Theca, Granulosa, and/or Leydig cells.

91. The method of any one of claims 86-90, wherein disrupting the development and/or function of somatic gonadal cells comprises disrupting at least one signaling molecule produced by Sertoli, Theca, Granulosa, and/or Leydig cells.

92. The method of any one of claims 86-91, wherein disrupting the development and/or function of somatic gonadal cells comprises disrupting at least one secreted diffusible signal protein or growth factor protein that regulates Spermatogonial stem cell (SCC) and/or Oogonial stem cell (OSC) renewal and/or differentiation.

93. The method of claim 92, wherein the at least one secreted diffusible signal protein or growth factor protein is glial cell line-derived neurotrophic factor (GDNF); bone morphogenetic protein 4 (BMP4); stem cell factor (SCF); fibroblast growth factor 2 (FGF2); C-X-C motif chemokine 12 (CXCL12); and/or epidermal growth factor (EGF).

94. The method of any one of claims 86-93, wherein the mutation that is present in the germline and disrupts the development and/or function of somatic gonadal cells is a mutation in microRNA miR-202.

95. The method of any one of claims 86-94, wherein the at least one germ cell is from about 500 germ cells to about 6,000 germ cells.

96. The method of any one of claims 86-95, wherein the at least one germ cell is transplanted into the peritoneal cavity of the embryo or hatchling of the endogenous germ cell-less fish, crustacean, or mollusk.

97. The method of any one of claims 86-96, wherein the endogenous germ cell-less fish, crustacean, or mollusk has a null mutation in dnd1, Elavl2, vasa, nanos3, and/or piwi-like gene.

98. The method of any one of claims 86-96, wherein the male and/or female endogenous germ cell-less fish, crustacean, or mollusk has a null mutation in dnd1, Elavl2, vasa, nanos3, and/or piwi-like gene.

99. The method of any one of claims 86-96, wherein the endogenous germ cell-less fish, crustacean, or mollusk is created using ploidy manipulation, such as triploidy manipulation.

100. The method of any one of claims 86-96, wherein the endogenous germ cell-less fish, crustacean, or mollusk is created by hybridization.

101. The method of any one of claims 86-96, wherein the endogenous germ cell-less fish, crustacean, or mollusk is created using exposure to high levels of sex hormones.

102. The method of any one of claims 86-96, wherein the endogenous germ cell-less fish, crustacean, or mollusk is created using morpholinos.

103. The method of any one of claims 86-96, wherein the endogenous germ cell-less fish, crustacean, or mollusk is created using sterile hybrids.

104. The method of any one of claims 86-96, wherein the endogenous germ cell-less fish, crustacean, or mollusk is created using chemical exposure.

105. The method of any one of claims 86-104, wherein the fish, crustacean, or mollusk is Atlantic salmon, Rainbow Trout, Coho Salmon, tilapia, cobia, Seriola spp., Grouper, Snapper, barramundi, Sea Bream, Sea Bass, lumpfish, sturgeon, Litopeneus vannamei, Peneaus monadon, oysters, clams or mussels.

106. The method of any one of claims 86-105, wherein the fish, crustacean, or mollusk is tilapia.

107. The fish, crustacean, or mollusk produced by the method of any one of claims 86-106.

108. A method of generating a sterile sex-determined fish, crustacean, or mollusk, comprising the steps of:
breeding (i) an endogenous germ cell-less fish, crustacean, or mollusk of any one of claims 66-85 and 107 that is female with (ii) an endogenous germ cell-less fish, crustacean, or mollusk of any one of claims 66-85 and 107 that is male and from the same sex-determination system as the (i) female, to produce the sterile sex-determined fish, crustacean, or mollusk.

109. A method of generating a sterile sex-determined fish, crustacean, or mollusk, comprising the steps of:
breeding (i) an endogenous germ cell-less fish, crustacean, or mollusk produced by the method of any one of claims 86-106 that is female with (ii) an endogenous germ cell-less fish, crustacean, or mollusk produced by the method of any one of claims 86-106 that is male and from the same sex-determination system as the (i) female, to produce the sterile sex-determined fish, crustacean, or mollusk.

110. An endogenous germ cell-less fish, crustacean, or mollusk having a chimeric gonad, wherein the chimeric gonad comprises at least one transplanted: a) Oogonial stem cell (OSC) from a homogametic superfemale donor, such as WW, having a mutation that is present in the germline and disrupts the development and/or function of somatic gonadal cells; or b) Spermatogonial stem cell (SCC) from a homogametic supermale donor, such as YY, having a mutation that is present in the germline and disrupts the development and/or function of somatic gonadal cells.

111. The endogenous germ cell-less fish, crustacean, or mollusk of claim 110, wherein the endogenous germ cell-less fish, crustacean, or mollusk is homozygous wild type in the germline at the locus for the mutation that is present in the germline and disrupts the development and/or function of somatic gonadal cells.

112. The endogenous germ cell-less fish, crustacean, or mollusk of claim 110 or 111, wherein disrupting the development and/or function of somatic gonadal cells comprises disrupting gametogenesis.

113. The endogenous germ cell-less fish, crustacean, or mollusk of any one of claims 110-112, wherein disrupting the development and/or function of somatic gonadal cells comprises disrupting the development and/or function of testis and/or ovary cells.

114. The endogenous germ cell-less fish, crustacean, or mollusk of any one of claims 110-113, wherein disrupting the development and/or function of somatic gonadal cells comprises disrupting the development and/or function of Sertoli, Theca, Granulosa, and/or Leydig cells.

115. The endogenous germ cell-less fish, crustacean, or mollusk of any one of claims 110-114, wherein disrupting the development and/or function of somatic gonadal cells comprises disrupting at least one signaling molecule produced by Sertoli, Theca, Granulosa, and/or Leydig cells.

116. The endogenous germ cell-less fish, crustacean, or mollusk of any one of claims 110-115, wherein disrupting the development and/or function of somatic gonadal cells comprises disrupting at least one secreted diffusible signal protein or growth factor protein that regulates Spermatogonial stem cell (SCC) and/or Oogonial stem cell (OSC) renewal and/or differentiation.

117. The endogenous germ cell-less fish, crustacean, or mollusk of claim 116, wherein the at least one secreted diffusible signal protein or growth factor protein is glial cell line-derived neurotrophic factor (GDNF); bone morphogenetic protein 4 (BMP4); stem cell factor (SCF); fibroblast growth factor 2 (FGF2); C-X-C motif chemokine 12 (CXCL12); and/or epidermal growth factor (EGF).

118. The endogenous germ cell-less fish, crustacean, or mollusk of any one of claims 110-117, wherein the mutation that is present in the germline and disrupts the development and/or function of somatic gonadal cells is a mutation in microRNA miR-202.

119. The endogenous germ cell-less fish, crustacean, or mollusk of any one of claims 110-118, wherein the at least one germ cell is from about 500 germ cells to about 6,000 germ cells.

120. The endogenous germ cell-less fish, crustacean, or mollusk of any one of claims 110-119, wherein the at least one germ cell is transplanted into the peritoneal cavity of the embryo or hatchling of the endogenous germ cell-less fish, crustacean, or mollusk.

121. The endogenous germ cell-less fish, crustacean, or mollusk of any one of claims 110-120, wherein the endogenous germ cell-less fish, crustacean, or mollusk has a null mutation in dnd1, Elavl2, vasa, nanos3, and/or piwi-like gene.

122. The endogenous germ cell-less fish, crustacean, or mollusk of any one of claims 110-120, wherein the endogenous germ cell-less fish, crustacean, or mollusk is created using ploidy manipulation, such as triploidy manipulation.

123. The endogenous germ cell-less fish, crustacean, or mollusk of any one of claims 110-120, wherein the endogenous germ cell-less fish, crustacean, or mollusk is created by hybridization.

124. The endogenous germ cell-less fish, crustacean, or mollusk of any one of claims 110-120, wherein the endogenous germ cell-less fish, crustacean, or mollusk is created using exposure to high levels of sex hormones.

125. The endogenous germ cell-less fish, crustacean, or mollusk of any one of claims 110-120, wherein the endogenous germ cell-less fish, crustacean, or mollusk is created using morpholinos.

126. The endogenous germ cell-less fish, crustacean, or mollusk of any one of claims 110-120, wherein the endogenous germ cell-less fish, crustacean, or mollusk is created using sterile hybrids.

127. The endogenous germ cell-less fish, crustacean, or mollusk of any one of claims 110-120, wherein the endogenous germ cell-less fish, crustacean, or mollusk is created using chemical exposure.

128. The endogenous germ cell-less fish, crustacean, or mollusk of any one of claims 110-127, wherein the fish, crustacean, or mollusk is Atlantic salmon, Rainbow Trout, Coho Salmon, tilapia, cobia, Seriola spp., Grouper, Snapper, barramundi, Sea Bream, Sea Bass, lumpfish, sturgeon, Litopeneus vannamei, Peneaus monadon, oysters, clams or mussels.

129. The endogenous germ cell-less fish, crustacean, or mollusk of any one of claims 110-128, wherein the fish, crustacean, or mollusk is tilapia.

130. A method of generating an endogenous germ cell-less fish, crustacean, or mollusk, comprising the steps of:
transplanting at least one: a) Oogonial stem cell (OSC) from a homogametic superfemale donor, such as WW, having a mutation that is present in the germline and disrupts the development and/or function of somatic gonadal cells; or b) Spermatogonial stem cell (SCC) from a homogametic supermale donor, such as YY, having a mutation that is present in the germline and disrupts the development and/or function of somatic gonadal cells, into an endogenous germ cell-less fish, crustacean, or mollusk, producing a chimeric gonad.

131. The method of claim 130, wherein the superfemale, such as WW, is generated by:
transplanting at least one Oogonial stem cell (OSC) from a heterogametic female donor, such as WZ, having a mutation that is present in the germline and disrupts the development and/or function of somatic gonadal cells, into a male and a female endogenous germ cell-less fish, crustacean, or mollusk, producing a chimeric gonad;
breeding the male endogenous germ cell-less fish, crustacean, or mollusk having a chimeric gonad with the female endogenous germ cell-less fish, crustacean, or mollusk having a chimeric gonad; and
selecting a progeny that is homogametic by genotypic selection.

132. The method of claim 130, wherein the supermale, such as YY, is generated by:
transplanting at least one Spermatogonial stem cell (SCC) from a heterogametic male donor, such as XY, having a mutation that is present in the germline and disrupts the development and/or function of somatic gonadal cells, into a male and a female endogenous germ cell-less fish, crustacean, or mollusk, producing a chimeric gonad;
breeding the male endogenous germ cell-less fish, crustacean, or mollusk having a chimeric gonad with the female endogenous germ cell-less fish, crustacean, or mollusk having a chimeric gonad; and
selecting a progeny that is homogametic by genotypic selection.

133. The method of any one of claims 130-132, wherein the recipient fish, crustacean, or mollusk is homozygous wild type in the germline at the locus for the mutation that is present in the germline and disrupts the development and/or function of somatic gonadal cells.

134. The method of any one of claims 130-133, wherein disrupting the development and/or function of somatic gonadal cells comprises disrupting gametogenesis.

135. The method of any one of claims 130-134, wherein disrupting the development and/or function of somatic gonadal cells comprises disrupting the development and/or function of testis and/or ovary cells.

136. The method of any one of claims 130-135, wherein disrupting the development and/or function of somatic gonadal cells comprises disrupting the development and/or function of Sertoli, Theca, Granulosa, and/or Leydig cells.

137. The method of any one of claims 130-136, wherein disrupting the development and/or function of somatic gonadal cells comprises disrupting at least one signaling molecule produced by Sertoli, Theca, Granulosa, and/or Leydig cells.

138. The method of any one of claims 130-137, wherein disrupting the development and/or function of somatic gonadal cells comprises disrupting at least one secreted diffusible signal protein or growth factor protein that regulates Spermatogonial stem cell (SCC) and/or Oogonial stem cell (OSC) renewal and/or differentiation.

139. The method of claim 138, wherein the at least one secreted diffusible signal protein or growth factor protein is glial cell line-derived neurotrophic factor (GDNF); bone morphogenetic protein 4 (BMP4); stem cell factor (SCF); fibroblast growth factor 2 (FGF2); C-X-C motif chemokine 12 (CXCL12); and/or epidermal growth factor (EGF).

140. The method of any one of claims 130-139, wherein the mutation that is present in the germline and disrupts the development and/or function of somatic gonadal cells is a mutation in microRNA miR-202.

141. The method of any one of claims 130-140, wherein the at least one germ cell is from about 500 germ cells to about 6,000 germ cells.

142. The method of any one of claims 130-141, wherein the at least one germ cell is transplanted into the peritoneal cavity of the embryo or hatchling of the endogenous germ cell-less fish, crustacean, or mollusk.

143. The method of any one of claims 130-142, wherein the endogenous germ cell-less fish, crustacean, or mollusk has a null mutation in dnd1, Elavl2, vasa, nanos3, and/or piwi-like gene.

144. The method of any one of claims 130-143, wherein the endogenous germ cell-less fish, crustacean, or mollusk is created using ploidy manipulation, such as triploidy manipulation.

145. The method of any one of claims 130-144, wherein the endogenous germ cell-less fish, crustacean, or mollusk is created by hybridization.

146. The method of any one of claims 130-144, wherein the endogenous germ cell-less fish, crustacean, or mollusk is created using exposure to high levels of sex hormones.

147. The method of any one of claims 130-144, wherein the endogenous germ cell-less fish, crustacean, or mollusk is created using morpholinos.

148. The method of any one of claims 130-144, wherein the endogenous germ cell-less fish, crustacean, or mollusk is created using sterile hybrids.

149. The method of any one of claims 130-144, wherein the endogenous germ cell-less fish, crustacean, or mollusk is created using chemical exposure.

150. The method of any one of claims 130-149, wherein the fish, crustacean, or mollusk is Atlantic salmon, Rainbow Trout, Coho Salmon, tilapia, cobia, Seriola spp., Grouper, Snapper, barramundi, Sea Bream, Sea Bass, lumpfish, sturgeon, Litopeneus vannamei, Peneaus monadon, oysters, clams or mussels.

151. The method of any one of claims 130-150, wherein the fish, crustacean, or mollusk is tilapia.

152. The fish, crustacean, or mollusk produced by the method of any one of claims 130-151.

153. A method of generating a sterile sex-determined fish, crustacean, or mollusk, comprising the steps of:
breeding (i) an endogenous germ cell-less fish, crustacean, or mollusk of any one of claims 110-129 and 152 that is female with (ii) an endogenous germ cell-less fish, crustacean, or mollusk of any one of claims 110-129 and 152 that is male and from the same sex-determination system as the (i) female, to produce the sterile sex-determined fish, crustacean, or mollusk.

154. A method of generating a sterile sex-determined fish, crustacean, or mollusk, comprising the steps of:
breeding (i) an endogenous germ cell-less fish, crustacean, or mollusk produced by the method of any one of claims 130-151 that is female with (ii) an endogenous germ cell-less fish, crustacean, or mollusk of any one of claims 130-151 that is male and from the same sex-determination system as the (i) female, to produce the sterile sex-determined fish, crustacean, or mollusk.


SEQUENCE LISTING (24 sequences)

SEQ ID NO: 1 - DNA, 1119 bp, Oreochromis niloticus
  source 1..1119: mol_type = genomic DNA
caggtaattgctgccatggaaacacagctatccaatgggcccacttgcaacaacacaagcaacggtccttcaactatcacaaacaactgctcctcacctgtagagtcagggagcgtagaggacagtaaaactaacttgatagtcaactatctgcctcagaacatgacccaggaggaactgaagagtttgtttgggagcatcggagaaattgagtcctgtaaactagttcgagacaaaatcacagggcagagcctaggctatggatttgtgaattatgtggacccaaaggatgcagaaaaggccatcaataccttaaatggcttgagacttcagaccaaaaccatcaaggtttcctatgcgcgtccaagctccgcctccatcagagatgcaaatttatacgtcagtggcctgccaaaaactatgactcagaaggaactggagcagctcttctctcagtacggacgcattattacctcacgcattctggtggaccaggtgactggtgtttccagaggagttggcttcattcgttttgaccggcgagttgaggctgaggaggccatcaagggtctgaactgtcagaagccgcctggtgccaccgaacccattacagtcaagtttgcaaacaacccgagccaaaagaccagccaggcactgctgtcccagctctatcagtcacccaatcgaaggtacccaggacccctcgcacagcaggcacaacgcttcaggttggacaatctgctgaacatggcctacggagtcaaaagctctatggcagtattgtgtagcaggttctccccgatggccattgacggggtgaccagcttggctggcatcaacatcccggggcacgcgggcactggctggtgcatcttcgtctacaacctggctccggacgcagatgaaagcatcctttggcagatgttcgggccgtttggtgctgtcacaaacgtcaaggttatccgcgactttaacacaaacaagtgcaaaggatttggttttgtcaccatgactaattacgacgaggcagctgtggccatcgccagcttgaatggataccgccttggggacagagttctgcaagtgtcattcaaaaccaacaaaacacacaaagcctga

SEQ ID NO: 2 - protein, 372 aa, Oreochromis niloticus
QVIAAMETQLSNGPTCNNTSNGPSTITNNCSSPVESGSVEDSKTNLIVNYLPQNMTQEELKSLFGSIGEIESCKLVRDKITGQSLGYGFVNYVDPKDAEKAINTLNGLRLQTKTIKVSYARPSSASIRDANLYVSGLPKTMTQKELEQLFSQYGRIITSRILVDQVTGVSRGVGFIRFDRRVEAEEAIKGLNCQKPPGATEPITVKFANNPSQKTSQALLSQLYQSPNRRYPGPLAQQAQRFRLDNLLNMAYGVKSSMAVLCSRFSPMAIDGVTSLAGINIPGHAGTGWCIFVYNLAPDADESILWQMFGPFGAVTNVKVIRDFNTNKCKGFGFVTMTNYDEAAVAIASLNGYRLGDRVLQVSFKTNKTHKA

SEQ ID NO: 3 - DNA, 525 bp, Oreochromis niloticus
  source 1..525: mol_type = genomic DNA
caggtccgaacactatttgtcagtgggctaccactggatattaaaccgcgggagctctacctcctcttcagaccatttaagggctatgaaggctccttgataaagctcacttctaaacagccagtggggtttgtcagttttgacagtcgatcagaggcggaggctgctaagaatgccttgaacggggtacgatttgacccagagattccccagactctgcggctggagttcgccaaggccaacaccaagatggccaaaaacaagctggttggcactcccaaccccccaccttctcagcagagccccgggccacagttcataagcagagacccatatgagctcacagtgcctgctctctatcccagcagcccagacgtgtgggcctcatacccgctgtacccggcggagctgtcgccggcccttccacccgctttcacctacccctcctcgctccacgctcagattcgttggctcccgcctgcagatggaactcctcagggatggaagtccaggcagttctgctga

SEQ ID NO: 4 - protein, 174 aa, Oreochromis niloticus
QVRTLFVSGLPLDIKPRELYLLFRPFKGYEGSLIKLTSKQPVGFVSFDSRSEAEAAKNALNGVRFDPEIPQTLRLEFAKANTKMAKNKLVGTPNPPPSQQSPGPQFISRDPYELTVPALYPSSPDVWASYPLYPAELSPALPPAFTYPSSLHAQIRWLPPADGTPQGWKSRQFC

SEQ ID NO: 5 - DNA, 120 bp, Oreochromis niloticus
  source 1..120: mol_type = genomic DNA
caggtaattgctgccatggaaacacagctatccaacttgcaacaacacaagcaacggtccttcaactatcacaaacaactgctcctcacctgtagagtcagggagcgtagaggacagtaa

SEQ ID NO: 6 - protein, 39 aa, Oreochromis niloticus
QVIAAMETQLSNLQQHKQRSFNYHKQLLLTCRVRERRGQ

SEQ ID NO: 7 - DNA, 239 bp, Oreochromis niloticus
  source 1..239: mol_type = genomic DNA
caggtccgaacactatttgtcagtgggctaccactggatattaaaccgcgggagctctacctcctcttcagaccatttaagggctatgaaggctccttgataaagctcacttctaaacagccagtggggtttgtcagttttgacagtcgatcagagtcgatcacacctacgatcggaggctgctaagaatgccttgaacggggtacgatttgacccagagattccccagactctgcggc

SEQ ID NO: 8 - protein, 61 aa, Oreochromis niloticus
QVRTLFVSGLPLDIKPRELYLLFRPFKGYEGSLIKLTSKQPVGFVSFDSRSESITPTIGGC

SEQ ID NO: 9 - DNA, 17 bp, Oreochromis niloticus
  source 1..17: mol_type = genomic DNA
gtatgtgcataggaaaa

SEQ ID NO: 10-11 - skipped (2 entries)

SEQ ID NO: 12 - DNA, 19 bp, Oreochromis niloticus
  source 1..19: mol_type = genomic DNA
tcctttttcctatgcacat

SEQ ID NO: 13 - DNA, 18 bp, synthetic construct
  source 1..18: mol_type = other DNA
tgtaaaacgacggccagt

SEQ ID NO: 14 - DNA, 18 bp, synthetic construct
  source 1..18: mol_type = other DNA
taggagtgcagcaagcat

SEQ ID NO: 15 - DNA, 21 bp, synthetic construct
  source 1..21: mol_type = other DNA
gttccagtgtccagaatcggg

SEQ ID NO: 16 - DNA, 18 bp, synthetic construct
  source 1..18: mol_type = other DNA
ctggtggaatacctctgc

SEQ ID NO: 17 - DNA, 87 bp, Oreochromis niloticus
  source 1..87: mol_type = genomic DNA
ctcgctgttcctatgcacatacttctttgagatttaactttaaagaggcataaggcatgggaaaatggggctgcagaggtattccac

SEQ ID NO: 18 - DNA, 86 bp, Oreochromis niloticus
  source 1..86: mol_type = genomic DNA
ctcgctgttccttgcacatacttctttgagatttaactttaaagaggcataaggcatgggaaaatggggctgcagaggtattccac

SEQ ID NO: 19 - DNA, 75 bp, Oreochromis niloticus
  source 1..75: mol_type = genomic DNA
ctcgctgtacttctttgagatttaactttaaagaggcataaggcatgggaaaatggggctgcagaggtattccac

SEQ ID NO: 20 - DNA, 1653 bp, Oreochromis niloticus
  source 1..1653: mol_type = genomic DNA
agacaatgcacaataggttacaaaaaagtttaaaagcagtcctccatacacagccgtttggtatttgtgacaaaatttcattccataccttagcgacgggctatgctaggccccgcccacggctcagtgggcactaaagacatagcatcgagtgtacgctggactactgcagttggaaacgggctacaaagtggcgtcgctgtgcgcacaaacacgctgagacgatggaaaacacgcaaagccaggtgctgaaccttgaacgggtgcaggccctggaaatctggttgaaagcaaccaacacaaagctgactcaagttaatggccagaggaaatatggaggaccacctgaggtgtgggaaggtcccacaccgggaccgcgctgtgaagtcttcatcagccagatcccacgggacacgtatgaggacatccttattcccctgttcagctccattgggccactctgggagttccggctgatgatgaacttcagtgggcagaaccgcggctttgcgtatgccaaatatggctcagctgctatagctgttgaagccatacgacagctgcacggtcacatggtggagcctggctaccgcatcagtgtacggcggagcacagagaagcgacacctttgtattggaggtctgcctgcttccactagacaagaaggcatactgcaggtgctgcgtatgctggtagagggggtggagagagtttccctgaaggccggacctggtatagagggggtatctgctactgttgctttctcatctcaccatgcagcttctatggctaagaaagtgctggtggaagcatttaagaagcagtttgcaatgtgtgtgtcagtcaagtggcagccaacagagaagccaaaccctgacgagccacgatgccctcagaaacgtgcaaagagcctgttgccgtcacacctagggcccctgcaccacagttctccacaaccctcaggcccgccttcattcctgaccctccctgcatccatacccgcaggtttctgcagagcagtgggagggcccactgctcctcagctcgctcaccctacatgctcttttcccaattcctccacccaaggccatcttgtatttgcagcatccccagtgatgcttctcagtgcagatccgcgggatcactgccgctttcaaggggttggtcatgatcttaccgggtcctaatgccagcaccatgctagaggaggctcagaaggctgtagcccagcaggtcctgcagaagatgtacaacactggtctcacacactaaacagctgatgccgtcctgcagttctgtttcaccttgtttgtgttatgtggtttcattttctgcatgtttttactagagtagcaccaagtttgtttctctgactataacttgtggtttgttttatgcatgatttttactgtacattagtgttctgtgttactggattggttctcattttaattaaatgagctttgaaaagaaagtgtcggcgtttctttcaaattaatgaaagatttaaattaacttaggaaaatggtaaagcagttattattgtctcacttcatgctgttatgaaccctagtgattctcatccagacctttacgtatctttgaaggttgtggattgagactaacccccctcagtggtttggcattttaaac

SEQ ID NO: 21 - protein, 320 aa, Oreochromis niloticus
MENTQSQVLNLERVQALEIWLKATNTKLTQVNGQRKYGGPPEVWEGPTPGPRCEVFISQIPRDTYEDILIPLFSSIGPLWEFRLMMNFSGQNRGFAYAKYGSAAIAVEAIRQLHGHMVEPGYRISVRRSTEKRHLCIGGLPASTRQEGILQVLRMLVEGVERVSLKAGPGIEGVSATVAFSSHHAASMAKKVLVEAFKKQFAMCVSVKWQPTEKPNPDEPRCPQKRAKSLLPSHLGPLHHSSPQPSGPPSFLTLPASIPAGFCRAVGGPTAPQLAHPTCSFPNSSTQGHLVFAASPVMLLSADPRDHCRFQGVGHDLTGS

SEQ ID NO: 22 - DNA, 1200 bp, Oreochromis niloticus
  source 1..1200: mol_type = genomic DNA
agacaatgcacaataggttacaaaaaagtttaaaagcagtcctccatacacagccgtttggtatttgtgacaaaatttcattccataccttagcgacgggctatgctaggccccgcccacggctcagtgggcactaaagacatagcatcgagtgtacgctggactactgcagttggaaacgggctacaaagtggcgtcgctgtgcgcacaaacacgctgagacgatggaaaacacgcaaagccaggtgctgaaccttgaacgggtgcaggccctggaaatctggttgaaagcaaccaacacaaagctgactcaagttaatggccagaggaaatatggaggaccacctgaggtgtgggaaggtcccacaccgggaccgcgctgtgaagtcttcatcagccagatcccacgggacacgtatgaggacatccttattcccctgttcagctccattgggccactctgggagttccggctgatgatgaacttcagtgggcagaaccgcggctttgcgtatgccaaatatggctcagctgctatagctgttgaagccatacgacagctgcacggtcacatggtggagcctggctaccgcatcagtgtacggcggagcacagagaagcgacacctttgtattggaggtctgcctgcttccactagacaagaaggcatactgcaggtgctgcgtatgctggtagagggggtggagagagtttccctgaaggccggacctggtatagagggggtatctgctactgttgctttctcatctcaccatgcagcttctatggctaagaaagtgctggtggaagcatttaagaagcagtttgcaatgtgtgtgtcagtcaagtggcagccaacagagaagccaaaccctgacgagccacgatgccctcagaaacgtgcaaagagcctgttgccgtcacacctagggcccctgcaccacagttctccacaaccctcaggcccgccttcattcctgaccctccctgcatccatacccgcaggtttctgcagagcagtgggagggcccactgctcctcagctcgctcaccctacatgctcttttcccaattcctccacccaaggccatcttgtatttgcagcatccccagtgatgcttctcagtgcagatccgcgggatcactgccgctttcaaggggttggtcatgatcgggtcctaatgccagcaccatgctaga

SEQ ID NO: 23 - protein, 324 aa, Oreochromis niloticus
MENTQSQVLNLERVQALEIWLKATNTKLTQVNGQRKYGGPPEVWEGPTPGPRCEVFISQIPRDTYEDILIPLFSSIGPLWEFRLMMNFSGQNRGFAYAKYGSAAIAVEAIRQLHGHMVEPGYRISVRRSTEKRHLCIGGLPASTRQEGILQVLRMLVEGVERVSLKAGPGIEGVSATVAFSSHHAASMAKKVLVEAFKKQFAMCVSVKWQPTEKPNPDEPRCPQKRAKSLLPSHLGPLHHSSPQPSGPPSFLTLPASIPAGFCRAVGGPTAPQLAHPTCSFPNSSTQGHLVFAASPVMLLSADPRDHCRFQGVGHDRVLMPAPC

SEQ ID NO: 24 - DNA, 94 bp, Oreochromis niloticus
  source 1..94: mol_type = genomic DNA
ctcgctgttcctttttcctatgcacatacttctttgagatttaactttaaagaggcataaggcatgggaaaatggggctgcagaggtattccac